(19) United States
(12) Patent Application Publication
(10) Pub. No.: US 2010/0221254 A1
(43) Pub. Date: Sep. 2, 2010

(54) Methods and Compositions for PDGF-C Activation and Inhibition

(75) Inventors: Linda Fredriksson, Stockholm (SE); Hong Li, Stockholm (SE); Christina Fieber, Stockholm (SE); Xuri Li, Stockholm (SE); Ulf Eriksson, Stockholm (SE)

Correspondence Address: CROWELL & MORING LLP;INTELLECTUAL PROPERTY GROUP, P.O. BOX 14300, WASHINGTON, DC, 20044-4300, US

(73) Assignee: Ludwig Institute for Cancer Research Ltd., New York, NY (US)

(21) Appl. No.: 12/652,998

(22) Filed: Jan. 6, 2010

Related U.S. Application Data
(62) Division of application No. 11/985,984, filed on Nov. 19, 2007, now abandoned.
Application No. 11/985,984 is a continuation of application No. 10/971,705, filed on Oct. 25, 2004, now abandoned.
(60) Provisional application No. 60/513,543, filed on Oct. 24, 2003.
(60) Provisional application No. 60/548,866, filed on Mar. 2, 2004.

(51) Int. Cl. A61K 39/395; C07K 16/00; A61P 35/00; A61P 9/10

(52) U.S. Cl. 424/139.1; 424/158.1; 530/389.2; 530/387.9; 530/388.23; 530/387.3

(57) ABSTRACT

Methods for inhibiting angiogenesis comprising administering tissue-plasminogen activator (tPA) inhibitors, and pharmaceutical compositions suitable for the methods comprising the tPA inhibitors. Also provided are methods for stimulating angiogenesis comprising administering tPA to a patient in need thereof, and pharmaceutical compositions comprising an effective amount of tPA for the methods of stimulation. The present invention discloses that tPA is a specific PDGF-C activating protease, and that the CUB-domains in PDGF-CC directly interact with the protease, are required for efficient proteolysis, and released CUB-domains are tPA inhibitors. Preferably, the method and compositions of the present invention are used for simultaneously stimulating, or simultaneously inhibiting, thrombolysis and angiogenesis.

### CROSS REFERENCE TO RELATED APPLICATIONS

The present application claims priority to the following provisional applications which are incorporated herein by reference in their entirety: U.S. Provisional Application No. 60/513,543, entitled “Methods and Compostions for PDGF-C Activation and Inhibition,” filed Oct. 24, 2003, and U.S. Provisional Application No. 60/548,866, entitled “Methods and Compostions for PDGF-C Activation and Inhibition,” filed Mar. 2, 2004.

### FIELD OF THE INVENTION

This invention relates to methods and compositions for activating or inhibiting a platelet-derived growth factor (PDGF), specifically PDGF-C. The invention is based on the discovery that the tissue-plasminogen activator (tPA) is a specific PDGF-C activating protease.

### BACKGROUND OF THE INVENTION

Platelet-derived growth factors (PDGFs) are important for normal tissue growth and maintenance, and are also involved in several pathological conditions such as malignancies, atherosclerosis and fibrosis. PDGF signaling is critical for normal tissue growth and maintenance, and is mediated through two structurally related tyrosine kinase receptors, PDGFR-α and PDGFR-β. The PDGF family consists of disulfide-bonded dimers involving four polypeptide chains: the classical PDGF-A and PDGF-B chains, the newly discovered PDGF-C (Li et al., 2000), and PDGF-D chains (Bergsten et al., 2001; LaRochelle et al., 2001). Unique for PDGF-C and PDGF-D chains are that they share a two-domain organization not found within the classical PDGF chains, with an N-terminal CUB domain in front of the conserved growth factor domain.

PDGF-C is secreted from cells as a latent dimer, PDGF-CC and it is known that regulated proteolytic removal of the CUB domain is required before PDGF-CC and PDGF-DD can bind to and activate their cognate PDGFRs. Activated PDGF-C, like PDGF-A, signals through PDGFR-α homodimers, and activated PDGF-D through PDGFR-β homodimers, whereas PDGF-B binds to and activates both PDGFRs (Heldin and Westermark, 1999; Li and Eriksson, 2003). Other groups have demonstrated that both PDGF-C and PDGF-D are able to activate PDGFRα/β heterodimeric complexes as well (Cao et al., 2002; Gilbertson et al., 2001; LaRochelle et al., 2001). The PDGFs often function in a paracrine mode as they are frequently expressed in cells in close apposition to the PDGFR-expressing mesenchyme (Ataliotis and Mercola, 1997), and the expression of PDGF-C is widespread during embryonic development (Aase et al., 2002; Ding et al., 2000).

In tumor cells and in cell lines grown in vitro, co-expression of PDGFs and their receptors may also generate autocrine loops resulting in cellular transformation (Betsholtz et al., 1984; Bishop et al., 1998; Keating and Williams, 1988). For the novel PDGFs, PDGF-C and PDGF-D, the PDGF receptor-mediated signaling is further complicated by the requirement for proteolytic activation of the latent factors.

PDGF-C and PDGF-D have been reported to be potent transforming growth factors, however some discrepancies between the reported transforming abilities emphasize the importance in understanding the proteolysis underlying the activation of PDGF-C and PDGF-D (LaRochelle et al., 2002; Li et al., 2003; Zwerner and May, 2001).

It is well established that PDGF-C expression is widespread in both normal adult and embryonic tissues, as well as in several pathological conditions including tumors. In order to understand the physiological roles of PDGF-C-mediated signal transduction in these processes, it is important to understand how latent full-length PDGF-CC becomes proteolytically activated to generate a receptor agonist. Although there are reports indicating the involvement of serum-derived factors (Gilbertson et al., 2001 and LaRochelle et al, 2001), the protease(s) responsible for activation of the novel PDGFs remain elusive. It was previously shown that the relatively non-specific protease plasmin can be used to activate both PDGF-CC and PDGF-DD from their latent precursors (Bergsten et al., 2001; Li et al., 2000); however, given the wide substrate specificity of plasmin, this protease is unlikely to be a physiologically relevant protease in activation of the novel PDGFs. Elucidating the identity, localization, and regulation of this protease(s) will greatly enhance understanding of PDGF regulation in vivo. In addition, the role of the CUB domain has not been fully understood. Thus there is a need for elucidating the roles the CUB domain plays in vivo and the identity of the protease(s) involved in PDGF-C activation in vivo.

Tissue plasminogen activator (tPA) is a secreted serine protease with highly restricted substrate specificity. tPA is best characterized for its role in releasing the broad-specificity protease plasmin from the inactive zymogen plasminogen (Plg), which then digests the fibrin network of blood clots to form soluble products. Since the activity of tPA is substantially accelerated in the presence of fibrin (Hoylaerts et al, 1982; Ranby, 1982) thereby facilitating a localized generation of plasmin, tPA has been investigated as a potential thrombolytic agent. In fact, tPA is currently the only treatment of acute ischemic stroke approved by the FDA (The National Institute of Neurological Disorders and Stroke rtPA Stroke Study Group, 1995). Recently, there have been several reports suggesting that tPA plays normal and pathological roles that do not require plasminogen (Wu et al, 2000; Nicole et al, 2001; Yepes et al, 2002, 2003), but so far only one other substrate, apart from plasminogen, has been reported for tPA, that is, the NR1 subunit of the NMDA receptor (Nicole et al, 2001).

### SUMMARY OF THE INVENTION

The invention is based on the surprising discovery that tPA cleaves and activates latent dimeric PDGF-CC. This is a novel role for tPA, which is a secreted serine protease with restricted specificity, its best characterized role being to release the broad spectrum protease plasmin from inactive zymogen Plg.

According to one aspect, the invention provides a method for inhibiting proteolytic processing of PDGF-C or PDGF-CC in a mammal in need thereof, comprising administering to the mammal an effective amount of tPA inhibitor. Preferably, the tPA inhibitor is an anti-tPA antibody, a PDGF-C CUB domain or a PDGF-CC CUB domain.

In another embodiment, a therapeutic method is provided for tumor treatment in a mammal, wherein the tumor is lined by or contains endothelial cells, the method comprising inhibiting proteolytic processing of PDGF-C or PDGF-CC in the mammal. Preferably, the method comprises administering to said mammal an effective amount of tPA inhibitor. Preferred tPA inhibitors include an anti-tPA antibody, a PDGF-C CUB domain or a PDGF-CC CUB domain. The method of the present invention is particularly suitable for the treatment of hemangioendothelioma, an angiosarcoma or a lymphangioma.

The invention also relates to a therapeutic method for treating an inflammatory disease or an autoimmune disease in a mammal, wherein the inflammatory disease or autoimmune disease involves increased proliferation of endothelial cells or endothelia-related cells (such as mesangial cells), the method comprising inhibiting proteolytic processing of PDGF-C or PDGF-CC in the mammal. Preferably, the method comprises administering to said mammal an effective amount of tPA inhibitor, such as an anti-tPA antibody, a PDGF-C CUB domain or a PDGF-CC CUB domain. The method is especially suitable for the treatment of glomerulonephritis.

The instant invention additionally embraces a method for stimulating angiogenesis in a mammal in need thereof, the method comprising administering to the mammal an effective amount of a protease, preferably tPA, to promote proteolytic processing of PDGF-C or of PDGF-CC.

In a particularly advantageous embodiment, the present invention provides a method for stimulating both angiogenesis and thrombolysis in a mammal in need thereof, the method comprising administering to the mammal an effective amount of a protease to promote proteolytic processing of PDGF-C or of PDGF-CC. A preferred protease is tPA.

In another embodiment, the present invention provides a method for promoting wound healing, where stimulation of both angiogenesis and thrombolysis are desired. According to this embodiment, an effective amount of a tPA to promote proteolytic processing of PDGF-C or of PDGF-CC is administered to a patient in need thereof. For example, this method is suitable for treatment of ulcers commonly occurring in diabetic patients. Other proteases, especially serine proteases, are also suitable for use in this method.

Also provided are pharmaceucial compositions for inhibiting proteolytic processing of PDGF-C or PDGF-CC in a mammal in need thereof, which composition comprises an effective amount of tPA inhibitor, and a pharmaceutically suitable excipient. Many protease inhibitors are tPA inhibitors suitable for the present invention. For example, they include naturally occurring serine protease inhibitors, which are usually polypeptides and proteins which have been classified into families primarily on the basis of the disulfide bonding pattern and the sequence homology of the reactive site. Serine protease inhibitors, including the group known as serpins, have been found in microbes, in the tissues and fluids of plants, animals, insects and other organisms. At least nine separate, well-characterized proteins are now identified, which share the ability to inhibit the activity of various proteases. Several of the inhibitors have been grouped together, namely α₁-proteinase inhibitor, antithrombin III, antichymotrypsin, C1-inhibitor, and α2-antiplasmin. These inhibitors are members of the α₁-proteinase inhibitor class. Others include the protein α₂-macroglobulin, α₁-antitrypsin (AAT) and inter-alpha-trypsin inhibitor. In addition, as disclosed in U.S. Pat. No. 6,001,355, the seed of Erythrina Latissima (broad-leafed Erythrina) and other Erythrina species contains two proteinase inhibitors, referred as DE-1 and DE-3. DE-3 has the property of being an enzyme inhibitor of the Kunitz type and of being an inhibitor for trypsin, plasmin and tPA. U.S. Pat. No. 5,973,118 further discloses a recombinant ETI polypeptide which has a specific inhibitory activity for t-PA and t-PA derivatives. Other peptide serine protease inhibitors are disclosed in U.S. Pat. No. 5,157,019. In addition, U.S. Pat. Nos. 5,424,329 and 5,350,748 disclose staurosporine and other small molecule tPA inhibitors. Likewise, U.S. Pat. No. 5,869,455 discloses N-substituted derivatives; U.S. Pat. No. 5,861,380 protease inhibitors-keto and di-keto containing ring systems; U.S. Pat. No. 5,807,829 serine protease inhibitor-tripeptoid analogues; U.S. Pat. No. 5,801,148 serine protease inhibitors-proline analogues; U.S. Pat. No. 5,618,792 substituted heterocyclic compounds useful as inhibitors of serine proteases. These patents and PCT publications and others as listed infra are incorporated herein, in their entirety, by reference. Other equally advantageous molecules, which may be used instead of α₁-antitrypsin or in combination therewith are contemplated such as in WO 98/20034 disclosing serine protease inhibitors from fleas. Without limiting to this single reference one skilled in the art can easily and without undue experimentation adopt compounds such as in WO98/23565 which discloses aminoguanidine and alkoxyguanidine compounds useful for inhibiting serine proteases; WO98/50342 discloses bis-aminomethylcarbonyl compounds useful for treating cysteine and serine protease disorders; WO98/50420 cyclic and other amino acid derivatives useful for thrombin-related diseases; WO 97/21690 D-amino acid containing derivatives; WO 97/10231 ketomethylene group-containing inhibitors of serine and cysteine proteases; WO 97/03679 phosphorous containing inhibitors of serine and cysteine proteases; WO 98/21186 benzothiazo and related heterocyclic inhibitors of serine proteases; WO 98/22619 discloses a combination of inhibitors binding to P site of serine proteases with chelating site of divalent cations; WO 98/22098 a composition which inhibits conversion of pro-enzyme CPP32 subfamily including caspase 3 (CPP32/Yama/Apopain); WO 97/48706 pyrrolo-pyrazine-diones; WO 97/33996 human placental bikunin (recombinant) as serine protease inhibitor; WO 98/46597 complex amino acid containing molecule for treating viral infections and conditions disclosed hereinabove. Other compounds having serine protease inhibitory activity are equally suitable and effective, including but not limited to: tetrazole derivatives as disclosed in WO 97/24339; guanidinobenzoic acid derivatives as disclosed in WO 97/37969 and in U.S. Pat. Nos. 4,283,418; 4,843,094; 4,310,533; 4,283,418; 4,224,342; 4,021,472; 5,376,655; 5,247,084; and 5,077,428; phenylsulfonylamide derivatives represented by general formula in WO 97/45402; novel sulfide, sulfoxide and sulfone derivatives represented by general formula in WO 97/49679; novel amidino derivatives represented by general formula in WO 99/41231; other amidinophenol derivatives as disclosed in U.S. Pat. Nos. 5,432,178; 5,622,984; 5,614,555; 5,514,713; 5,110,602; 5,004,612; and 4,889,723 among many others.

Preferably, the pharmaceutical composition comprises an effective amount of tPA inhibitor for tumor treatment in a mammal, wherein the tumor is lined by or contains endothelial cells. Particularly preferably, the pharmaceutical composition is suitable for the treatment of hemangioendothelioma, angiosarcoma or lymphaangioma, or for the treatment of inflammatory diseases or autoimmune diseases in a mammal, wherein the inflammatory disease or autoimmune disease involves increased proliferation of endothelial cells or related cells, such as glomerulonephritis.

The present invention further provides a pharmaceutical composition for stimulating angiogenesis in a mammal in need thereof, comprising an effective amount of tPA to promote proteolytic processing of PDGF-C or of PDGF-CC, and a pharmaceutically acceptable excipient. In a preferred embodiment, the pharmaceutical composition is effective for stimulating both angiogenesis and thrombolysis in a mammal in need thereof.

A pharmaceutical composition of the invention contains tPA or its inhibitors (“active ingredients”), and an appropriate pharmaceutically acceptable carrier. The term “pharmaceutically acceptable carrier” refers to those solid and liquid substances, which do not significantly or adversely affect the therapeutic properties of the peptides. Suitable pharmaceutical carriers are described in Remington's Pharmaceutical Sciences 1990, pp. 1519-1675, Gennaro, A. R., ed., Mack Publishing Company, Easton, Pa. The serine protease inhibitor molecules of the invention can be administered in liposomes or polymers (see, Langer, R. Nature 1998, 392, 5).

The active ingredients may be administered as free chemicals or pharmaceutically acceptable salts thereof. The terms used herein conform to those found in Budavari, Susan (Editor), “The Merck Index” An Encyclopedia of Chemicals, Drugs, and Biologicals; Merck & Co., Inc. The term “pharmaceutically acceptable salt” refers to those acid addition salts or metal complexes which do not significantly or adversely affect the therapeutic properties (e.g. efficacy, toxicity, etc.).

The pharmaceutical compositions of the present invention may be administered to individuals, particularly humans, either intravenously, subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of inhibitors of serine proteases. Transdermal administration is accomplished by application of a cream, rinse, gel, etc. capable of allowing the inhibitors of serine proteases to penetrate the skin and enter the blood stream. Parenteral routes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy, tracheostomy, or endotracheal tube. In addition, osmotic pumps may be used for administration. The necessary dosage will vary with the particular condition being treated, method of administration and rate of clearance of the molecule from the body.

The compositions may, where appropriate, be conveniently presented in discrete unit dosage forms and may be prepared by any of the methods well known in the art of pharmacy. Pharmaceutical compositions suitable for oral administration may be presented as discrete unit dosage forms such as hard or soft gelatin capsules, cachets or tablets, each containing a predetermined amount of the active ingredient; as a powder or as granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art., e.g., with enteric coatings.

Oral liquid preparations may be in the form of, for example, aqueous or oily suspension, solutions, emulsions, syrups or elixirs, or may be presented as a dry product for constitution with water or another suitable vehicle before use. Such liquid preparations may contain conventional additives such as suspending agents, emulsifying agents, non-aqueous vehicles (which may include edible oils), or preservative.

The compounds may also be formulated for parenteral administration (e.g., by injection, for example, bolus injection or continuous infusion) and may be presented in unit dose form in ampoules, pre-filled syringes, small bolus infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in Fisher et al. (U.S. Pat. No. 4,788,603) or Bawas et al. (U.S. Pat. Nos. 4,931,279, 4,668,504 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. Nos. 4,140,122, 4,383,529, or 4,051,842. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention may also contain other adjuvants such as flavorings, coloring, antimicrobial agents, or preservatives.

The invention particularly relates to antagonists, such as antibodies or small molecules, that target the site of proteolysis in PDGF-C. A peptide sequence, either a monomer or a dimer, which includes the site of PDGF-C proteolysis can be used as an immunogen for generation of antibodies. The antibodies could be polyclonals, monoclonals, or bispecific antibodies recognizing the PDGF-C proteolytic site and another target eg. PDGF-D proteolytic site. Preferably, the antibodies would be chimerised, humanized or fully human. They could be F(ab)2 fragments, or single chain antibodies or single domain antibodies. Such antibodies and small molecules essentially protect the site of PDGF-C proteolysis by binding to it and thereby preventing tPA binding and subsequent cleavage. The immunogen could also be a fusion protein of the proteolyic site and another immunogen.

A preferred target for the antagonist comprises amino acids 231-234 of PDGF-C, especially preferably amino acids 231-235 of PDGF-C. However any antibody or small molecule which binds to any 4 or 5 consecutive amino acids within the range from amino acid 228 to amino acid 238 of PDGF-C could function as an effective antagonist to prevent proteolytic cleavage of PDGF-C.

Small molecule screening could use a library of PDGF-C fragments as substrate or the full-length PDGF-C. It is also within the scope of the invention to screen antibodies and small molecules for agonistic effects, i.e., as promoters of proteolysis.

Another class of substances that serve as inhibitors of PDGF-C or PDGF-CC activation by tPA is aptamers, which can be selected via the Systematic Evolution of Ligands by Exponential Enrichment (SELEX) process. SELEX is a method for the in vitro evolution of nucleic acid molecules with highly specific binding to target molecules and is described in e.g. U.S. Pat. Nos. 5,475,096, 5,580,737, 5,567,588, 5,707,796, 5,763,177, 6,011,577, and 6,699,843, incorporated herein by reference in their entirety. An aptamer has a unique sequence, has the property of binding specifically to a desired target compound, and is a specific ligand of a given target compound or molecule. The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets. Because the specific tPA proteolysis site on PDGF-C and PDGF-CC is known, screening using the SELEX process for aptamers that act on either PDGF-C/PDGF-CC or tPA would allow the identification of aptamers that inhibit tPA proteolysis of PDGF-C or PDGF-CC. The SELEX method involves selection from a mixture of candidate oligonucleotides and step-wise iterations of binding, partitioning and amplification, using the same general selection scheme, to achieve desired binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule.

The invention also relates to a molecule comprising a PDGF-C CUB domain or analog which functions as an inhibitor of PDGF-C proteolysis. Such CUB domain molecules (including allelic variants and hybridizing sequences) bind tPA so that the tPA is sequestered away from the full length PDGF-C and thus cannot bring about the proteolytic cleavage of the full length PDGF-C protein.

The invention further relates to a method of treating conditions involving undesired fibrinolysis in a patient, said method comprising administering a therapeutically effective amount of tPA inhibitor, such as a CUB domain molecule to a patient in need thereof, whereby the tPA inhibitor, e.g., a CUB domain molecule, binds tPA and inhibits fibrinolysis.

Another aspect of the invention relates to combined antagonism of proteolysis and inhibition of downstream signalling from the receptor. Blocking proteolysis of the full length PDGF-C prevents formation of the processed or mature form of PDGF-C which binds to the PDGFR-α and thereby inhibits downstream signalling.

In addition, the invention also relates to antagonists for “hemi-dimers” which comprise dimers formed between an unprocessed, full length PDGF-C molecule and a processed, mature form of the molecule, and to a method for inhibiting the activity of such hemi-dimers comprising administering a suitable antagonist.

Antibodies used in the invention are preferably chimeric or humanized or fully human antibodies. The antagonists useful in the invention also may include various fragments of immunoglobulin or antibodies known in the art, i.e., Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is a multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain moiety or, a single chain polypeptides containing only one heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof. A fragment of an antinody or immunoglobulin can also have bispecific function as described below.

The antagonists may also be bispecific antibodies, which are monoclonal, preferably human or humanized, antibodies that have binding specificities for at least two different antigens. In the present case, one of the binding specificities is for tPA and the other one is for any other antigen, and preferably for a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. It is also well known within the art of how to generate bispecific antibodies, or bispecific antibody fragments, by using recombinant DNA techniques (Kriangkum et al. Biomol Eng. 2001 September; 18(2):31-40).

Suitable antagonists thus may comprise an antibody, an Fv fragment, an F_{c} fragment, an F_{d} fragment, a Fab fragment, a Fab′ fragment, a F(ab)₂ fragment, F(ab′)₂ fragment, an scFvs fragment, a single chain antibody, a multimeric antibody, or any combination thereof. If desired, the immunoglobulin molecule may be joined to a reporter or chemotherapeutic molecule, or it may be joined to an additional fragment, and it may be a monomer or a multimeric product. The immunoglobulin molecule may also be made recombinantly, to include all or part of the variable regions and/or CDRs.

The above methods and compositions are especially suitable for use in human treatment.

### BRIEF DESCRIPTION OF THE DRAWINGS

FIG. 1 shows the characterization of a PDGF-CC processing activity. (A) Endogenous expression of PDGF-CC from AG1523 fibroblasts detected by a PDGF-C-specific antibody. Reduced latent PDGF-C migrated as a 48 kDa species, while the released core domain migrated as a 22 kDa species. (B) Using an anti-His₆ antibody, immunoreactivity was detected only in recombinant latent PDGF-C expressed in baculovirus-infected cells and not in conditioned medium from AG1523 cells. (C) Increasing concentrations of conditioned medium from AG1523 cells were incubated with fixed amounts of recombinant latent PDGF-CC. The reduced (R) and nonreduced (NR) recombinant proteins were analyzed by immunoblotting using an anti-His₆ antibody. Under reducing conditions, the 48 kDa latent PDGF-C and the released 22 kDa core domain of PDGF-C were visualized. Under nonreducing conditions, the 90 kDa latent homodimer of PDGF-CC, the 60 kDa hemidimer, and the 35 kDa homodimeric core domain of PDGF-CC were visualized. (D) Quantification of the amounts of reduced full-length 48 kDa (▪) and cleaved 22 kDa (♦) PDGF-C species. The results are mean±s.d. of five independent experiments. (E) Different protease inhibitors were preincubated with AG1523 medium, and then incubated with recombinant full-length PDGF-CC. Recombinant PDGF-CC incubated with serum-free medium (control) or AG1523 medium only (−) were used as controls. All lanes with incubations pretreated with serine protease inhibitors displayed reduced PDGF-C processing activity. An anti-His₆ antibody was used. (F) List of the protease inhibitors used and the specificity of the inhibitors.

FIG. 2 shows the cloning of candidate proteases from AG1523 fibroblastic cells. (A) Agarose gel electrophoresis of PCR products (arrowheads) amplified from AG1523 cDNA using degenerate oligonucleotide mixtures derived from trypsin-like serine protease domains. The amplified PCR fragments were cloned into the pCR2.1-TOPO vector and the nucleotide sequences of 18 clones were determined. (B) Histogram showing the identification of candidate proteases and distribution of the sequenced PCR-generated clones obtained from AG1523 cells.

FIG. 3 shows that tPA specifically cleaves latent PDGF-CC, using a coexpression and functional analysis of tPA and neurotrypsin (NT) on the proteolysis of PDGF-CC and PDGF-DD. (A, B, E) COS-1 cells were transfected with combinations of expression vectors encoding for PDGF-C or PDGF-D and different concentrations encoding for tPA and NT, respectively. Empty vector (mock) and the expression vectors alone were used as negative control. When coexpressed with PDGF-C, tPA released a 22 kDa fragment of PDGF-C (A, arrow), while tPA did not release the corresponding part of PDGF-D (B). In transfected cells, coexpressing NT and PDGF-C or PDGF-D, or mock transfection, did not release the core domains of PDGF-CC nor PDGF-DD; (C, D) In vitro cleavage of recombinant PDGF-CC (C) and PDGF-DD (D) using purified tPA in two different concentrations. PDGF-CC, but not PDGF-DD, is readily cleaved by tPA generating a 22 kDa band under reducing conditions, corresponding to the released core domain (lower arrowhead in C). Note the intermediate 32 kDa PDGF-C species (C, upper arrowhead), possibly due to cleavage by plasmin contamination in the tPA preparation; (E) Addition of the specific plasmin inhibitor a2-anti-plasmin (a2AP) into the cotransfection medium had no effect on the release of core PDGF-C by tPA nor had removal of Plg from the culture medium. N, normal FCS medium; D, Plg-depleted FCS medium.

FIG. 4 shows that tPA is the major PDGF-CC processing protease secreted from AG1523 cells and from primary mouse fibroblasts in culture. (A) Inhibition of cleavage of endogenous PDGF-CC produced by AG1523 cells using aprotinin and different concentrations of the specific tPA inhibitor tPA-STOP™. The inhibitors blocked processing of latent PDGF-CC showing that tPA accounts for the majority of the PDGF-C processing activity in conditioned media from AG1523 cells. (B) Serum-free media from wild-type and tPA-deficient fibroblasts were analyzed by immunoblotting. The results showed that both wild-type (+/+) and tPA-deficient (−/−) cells expressed latent PDGF-CC. However, tPA-deficient cells displayed a greatly reduced ability to process and activate the latent growth factor. tPA expression was analyzed by immunoblotting of conditioned media (middle panel). Agarose gel electrophoresis of PCR reactions from the genotyping of the animals used to establish the primary cultures of fibroblasts (lower panel). The immunoblot analyses were performed using protein-specific antibodies.

FIG. 5 shows that tPA-mediated proteolysis of latent PDGF-CC generates a PDGFR-α agonist. Conditioned serum-free media from transfected COS-1 cells were used to induce tyrosine phosphorylation of PDGFR-α expressed in PAE cells. (A) The 22 kDa fragment of PDGF-C, generated by tPA-mediated cleavage of latent PDGF-CC, induced efficient tyrosine phosphorylation of PDGFR-α as compared to mock, tPA, and PDGF-C controls as analyzed using antibodies against phosphotyrosine (PY99) (upper panel). The amount of precipitated PDGFR-α was monitored using antibodies to PDGFR-α (CED, middle panel). The amount of PDGF-C core domain in the media from the transfected cells was monitored by immunoblotting (lower panel). (B) Direct interaction of PDGF-CC with tPA. Ni-NTA beads coated with recombinant Hiss-tagged latent PDGF-CC, CUB domain, and core domains of PDGF-CC, or latent PDGF-DD, were incubated with purified tPA. Proteins eluted from the beads using a buffer containing 400mM imidazole were analyzed by immunoblotting using specific antibodies. The results show that latent PDGF-CC interacts directly with tPA both via the CUB and the core domains. (C) Illustration of the cleavage site mutant. (D) Analysis of the cleavage site mutant of PDGF-CC using the cotransfection assay. Normal and mutant latent PDGF-CC forms were expressed in transfected COS-1 cells, without or with the coexpression of tPA. Analysis by immunoblotting showed that cleavage of latent PDGF-CC by tPA was abolished in the alanine cleavage site mutant (upper panel) suggesting that the tribasic site is the cleavage site for tPA. The expression of tPAwas also monitored (lower panel).

FIG. 6 shows that the CUB domain of PDGF-C is required for the proteolysis of PDGF-CC with tPA. (A) Illustration of the mutant proteins used to determine the structural requirements of PDGF-CC for proteolytic activation by tPA. The corresponding expression constructs were transfected into COS-1 cells in the absence (B) or presence (C) of co-expressed tPA. tPA released a 22 kDa fragment only when co-expressed with full-length PDGF-CC. The PDGF-C species were detected by immunoblotting using a specific antibody to the core domain, and tPA expression was monitored using a polyclonal antibody against tPA (C, lower panel). (D) The N-terminally truncated variants of PDGF-CC were able to stimulate PDGFR-α activation. The relative amount of recombinant PDGF-C deletion proteins in the conditioned media was determined by enzyme-linked immunosorbent assay (ELISA) before addition to the PDGFR-α cells. Unstimulated PAE cells (−), cells stimulated with recombinant PDGF-BB (PB), recombinant core PDGF-CC (core PC) and conditioned medium from mock transfected cells were used as controls.

FIG. 7 demonstrates that an autocrine tPA-dependent growth stimulatory loop involving activation of latent PDGF-CC drives proliferation of fibroblasts in primary culture. Primary cultures of fibroblasts were established from wild-type and tPA-deficient animals. (A) Total cell numbers of wild-type (+/+) and tPA-deficient cells (−/−) after 36 h of culture in serum-free conditions (mean±s.d., n=4). Significantly less tPA-deficient cells were observed after the culture period (P<0.05). The tPA-deficient cells were stimulated to grow by the addition of activated PDGF-CC (mean±s.d., n=3). The seeding control was set to 100%. (B) Microphotographs showing wild-type and tPA-deficient fibroblasts following labeling with BrdU. Cell nuclei were visualized using DAPI (left column; blue), while BrdU-labeled nuclei were identified by immunofluorescence using a specific antibody (right column, red). (C) Quantification showed that significantly less tPA-deficient cells incorporated BrdU as compared to wild-type cells. Stimulation of the tPA-deficient cells with activated PDGF-CC or tPA enhanced BrdU incorporation, while wild-type cells were not markedly stimulated by this treatment (mean±s.d., n=3; n=2 for tPA treatment). *P<0.05, **P<0.01. (D) Activated PDGF-CC protein induced more efficient tyrosine phosphorylation of PDGFR-β in the tPA-deficient cells as compared to wild-type cells as analyzed using antibodies against phosphotyrosine (PY99) (upper panel). The amount of precipitated PDGFR-α was monitored using antibodies to PDGFR-α (CED, lower panel). These results show that growth of primary fibroblasts in culture is dependent on a growth stimulatory loop involving a tPA-dependent activation of latent PDGF-CC.

FIG. 8 shows colocalization of PDGF-CC and tPA. Immunohistochemical localization of PDGF-C (first column) and tPA (second column) in E14.5 mouse embryo and in T241 tumor xenografts. Tissue sections were stained using specific antibodies. (A, B) Developing kidney; overlapping staining for both PDGF-C and tPA was observed in the collecting ducts (cd). PDGF-C was also expressed in the collecting tubules (ct). (C, D) Skin of abdomen; colocalization of PDGF-C and tPA was seen in the germinal layer of the skin (gl) and in the surface ectoderm (se). (E, F) Expression of PDGF-C and tPA in T241 tumor xenografts. Scale bars, 50 mm.

FIG. 9 demonstrates that co-expression of “free” CUB domain of PDGF-C markedly reduces the cleavage of full-length PDGF-CC by tPA. The figure shows immunoblots of TCA-precipitated serum-free media from co-transfected COS-1 cells probed with antibodies to PDGF-C (PC_{core}), tPA, and anti-c-myc antibodies (to the CUB domain) (CUB_{c-myc}).

FIG. 10 shows hypothetical mechanisms involved in the activation of PDGF-CC by tPA. (A) tPA binds to both the CUB domain and the growth factor domain of latent PDGF-CC. Released CUB domains might act as competitive inhibitors of the subsequent proteolytic activation of PDGF-CC. (B) A tPA-mediated activation of latent PDGF-CC drives proliferation of primary fibroblasts in culture.

### DETAILED DESCRIPTION OF PREFERRED EMBODIMENTS

To identify the enzyme responsible for activation of latent PDGF-CC, the present inventors developed an in vitro assay to monitor cleavage of latent PDGF-CC, and by using a combination of protease inhibitor profiling (so-called reverse biochemistry; Takeuchi et al, 1999), molecular cloning with RT-PCR using degenerate primers, and a functional assay, tPA was identified as a specific protease able to activate latent PDGF-CC. Despite the close structural similarities between PDGF-C and PDGF-D, the latter factor was not activated by tPA, demonstrating that distinct pathways are involved in activation of the two factors.

tPA is a multidomain trypsin-like serine protease best known for its role in fibrinolysis via proteolytic activation of plasminogen into plasmin (for reviews, see Vassalli et al, 1991; Collen, 2001). However, the expression pattern of tPA in the mouse embryo, especially in neuronal tissue and in areas undergoing extensive tissue remodeling, suggests that the protease may serve additional functions (Rickles and Strickland, 1988; Carroll et al, 1994). Also, several reports have suggested that tPA plays normal and pathological roles that do not require plasminogen activation (Strickland, 2001; Tsirka, 2002), but apart from plasminogen, only one additional substrate has been identified, that is, the NR1 subunit of the NMDA receptor (Nicole et al, 2001). The identification of tPA as a specific activator of latent PDGF-CC is thus rather unexpected, but it provides additional evidence for roles of tPA in nonthrombolytic events, including fibrosis, angiogenesis, and tumor growth.

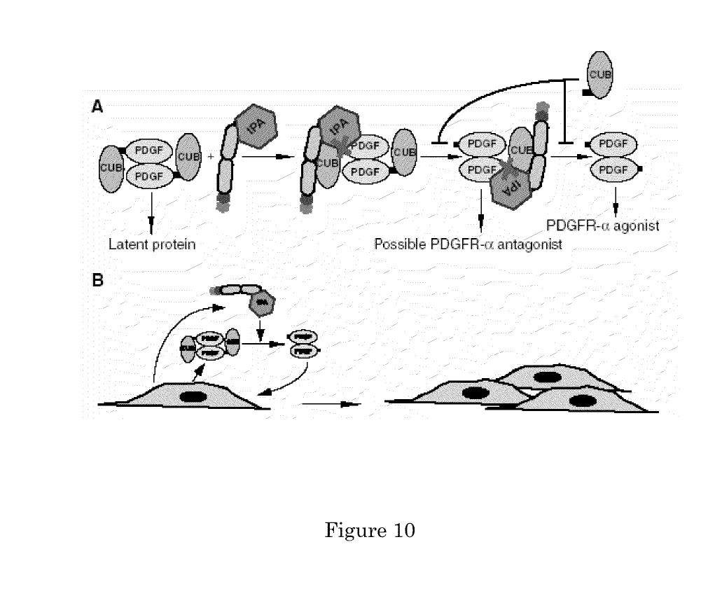

The mechanisms underlying the specific cleavage and activation of latent PDGF-CC by tPA involve the formation of a stable substrate-protease complex. The present disclosure shows that tPA specifically interacts with both the CUB and the PDGF/VEGF-like growth factor domain in PDGF-CC. The specific binding of tPA to the CUB domain of PDGF-C, and not that of PDGF-D, is required for proteolytic activation of the factor. Thus, the role of the CUB domain in PDGF-CC appears two-fold: to prevent an agonistic role of the unprocessed growth factor (Li et al, 2000) and to bind specifically tPA to allow a site-specific cleavage of the factor. CUB domains in different proteins are known to be involved in protein-protein interactions (e.g., see Thielens et al, 1999; Nakamura and Goshima, 2002). Thus, it is reasonable that the released CUB domains act as a competitive inhibitor in the activation of latent PDGF-CC. Although the structural domains of tPA interacting with the CUB domain of PDGF-C are unknown, FIG. 10A summarizes the findings of the present invention regarding the complex formation of full-length PDGF-CC and tPA, and the functional consequences of the growth factor when only one or both CUB domains have been removed by tPA-mediated proteolysis.

The tight complex formation of tPA and PDGF-CC allows a precise cleavage of the substrate. Previously, it was suggested that a conserved tribasic region (amino-acid residues -R231-K232-S233-R234- in human PDGF-C), 15 amino-acid residues N-terminal of the first cysteine in the PDGF/VEGF-like domain, represented a putative proteolytic cleavage site (Li et al, 2000). This suggestion was based on the location of this site in relation to the well-defined cleavage sites found in the intracellular proforms of PDGF-A and PDGF-B. The present invention verifies that the corresponding site in PDGF-C is the cleavage site for tPA.

The functional activity of tPA is tightly regulated and several stimuli including growth factors, cytokines, and metabolic conditions affect the synthesis and release of the enzyme. tPA is particularly abundant in vascular endothelial cells (van Hinsbergh et al., 1991; van Zonneveld et al., 1986a). In addition, the extracellular activity of tPA is controlled by plasminogen activator inhibitors (PAIS), and its enzymatic activity is strongly stimulated by fibrin peptides (van Zonneveld et al., 1986b). The multitude of factors controlling tPA availability and activity indicate that, PDGF-CC activation and subsequent initiation of PDGFR-mediated signal transduction are complex.

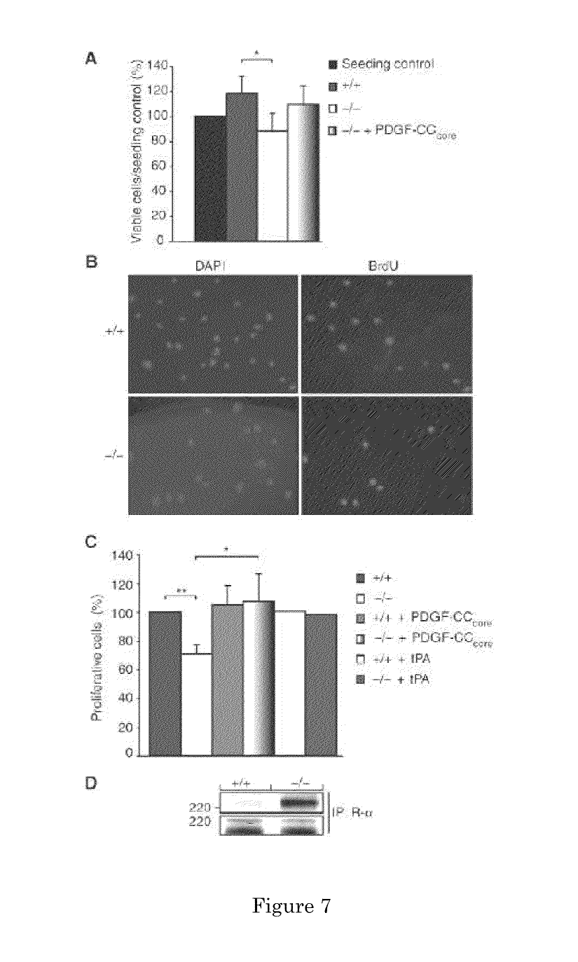

Components of the fibrinolytic system, including tPA, urokinase-type plasminogen activator (uPA), the urokinasetype plasminogen activator receptor (uPAR), and the plasminogen activator inhibitors (PAIS), are often overexpressed in tumors (Kwaan, 1992 and references therein). So far, strong evidence suggests that overexpression of uPA, uPAR, and PAIS is linked to increased tumor growth, invasion, and metastatic spreading, whereas less is known about the role of tPA in these processes. In addition, many types of tumors overexpress PDGF-C (Uutela et al, 2001; Zwerner and May, 2001; Andrae et al, 2002; Dijkmans et al, 2002; Lokker et al, 2002; U Eriksson, unpublished observation). According to the present invention, in PDGF-C-expressing tumors, tPA contributes to the activation of the growth factor. Several studies have shown that PDGF-C overexpression in tumor cells enhances tumor growth by promoting cellular transformation, and stimulates stromogenesis and tumor vascularization (Zwerner and May, 2001; Cao et al, 2002; Li et al, 2003). The source of tPA could either be PDGF-CC-expressing tumor cells themselves or as shown here for the T241 tumor the enzyme may be released by the invading endothelial cells of the tumor vasculature (FIG. 7F). Accordingly, inhibitors of tPA would also inhibit the growth of these tumors.

As indicated above, tPA administration is the only FDA-approved thrombolytic therapy for acute ischemic stroke, and increasing evidence from studies in animal models of embolic stroke cautions against the use of tPA, as it might mediate neuronal damage (Tsirka, 2002). At least part of the neuronal damage might be caused by a tPA-dependent, plasminogen-independent opening of the blood-brain barrier mediated via the low-density lipoprotein receptor-related protein (LRP) and the cleavage of an as yet unidentified substrate (Yepes et al, 2003). Interestingly, LRP is a negative regulator of PDGF signaling (Boucher et al, 2003), raising the possibility that part of the plasminogen-independent action of tPA is indeed mediated via modulation of PDGF signaling.

One drawback of using tPA in these conditions, compared to using other thrombolytic agents, is its ability to induce exitotoxin-induced neuronal degeneration and seizures (Tsirka et al., 1995; Wang et al., 1998). It was recently shown that activated PDGF-CC is a strong inducer of neoangiogenesis in a cornea pocket model (Cao et al., 2002). In models of experimentally induced ischemia of the heart and hind limb, systemic delivery of activated PDGF-CC promotes neoangiogeneis and tissue repair. At least in part the effects of PDGF-CC treatment in the ischemic models is caused by activation and recruitment of bone marrow-derived progenitor cells into the ischemic areas. According to an embodiment of this invention, tPA treatment of infarcted patients is able to activate endogenous latent PDGF-CC stores. Accordingly, the present invention provides methods of treatment with tPA that result in stimulation of therapeutic angiogensis along with the thrombolytic effects.

The finding by the present inventors that the growth of fibroblasts is dependent on a tPA-mediated activation of latent PDGF-CC, thus generating autocrine and paracrine growth stimulatory loops, indicates that PDGF-CC plays several roles in normal and pathological conditions involving fibroblast growth and recruitment. Such conditions include tissue morphogenesis and regeneration, wound healing, and tumor growth (see FIG. 10B). In part, this mechanism may also be the explanation for the long-standing observation that it is relatively easy to establish primary cultures of fibroblasts in comparison to most other cell types.

The present identification of tPA as a potent activator of latent PDGF-CC has provided novel insights into PDGF-mediated signaling with broad implications in normal and pathological conditions, in particular in tumor biology and cardiovascular medicine. The expression and proteolytic activity of tPA is regulated by many different factors and stimuli. One particularly interesting observation is that plasminogen activator inhibitor type 1 (PAI-1) controls the proteolytic activity of tPA. It is known that PAI-1 is upregulated by hypoxia (see e.g. Fink et al., 2002, Identification of a tightly regulated hypoxia-response element in the promoter of human plasminogen activator inhibitor-1. Blood. 99:2077-83). Accordingly, under hypoxia conditions, its proteolytic activities on tPA will also be increased. In other words, under hypoxia conditions, the proteolytic activity of tPA and thus processing and activation of PDGF-CC will be inhibited.

This may have bearings on angiogenesis and tissue repair in hypoxic conditions such as wound healing, and in particular healing of diabetic ulcers. It should be pointed out that diabetic patients often have an upregulation of PAI-1 (see e.g. Lyon et al., 2003, Effect of plasminogen activator inhibitor-1 in diabetes mellitus and cardiovascular disease. Am J Med. 115 Suppl 8A:62S-68S), presumably due to the microangiopathy that generate a slightly hypoxic state of many diabetic tissues.

Accordingly, the present invention provides methods for regulating tPA activities by way of regulating PAI-1 expression level or activity. Specifically, the method comprises administering a PAI-1 antagonist, such as an antibody, antisense nucleic acid molecule; or an RNAi molecule against a PAI-1 gene, or other known PAI-1 inhibiting small molecules, to a patient in need thereof. Preferably, the patient or the area of treatment is under hypoxic conditions. In a preferred embodiment, a PAI-1 antagonist is administered to the patent topically.

### Examples

### Example 1

### Identification and Cloning of a PDGF-CC Processing Protease

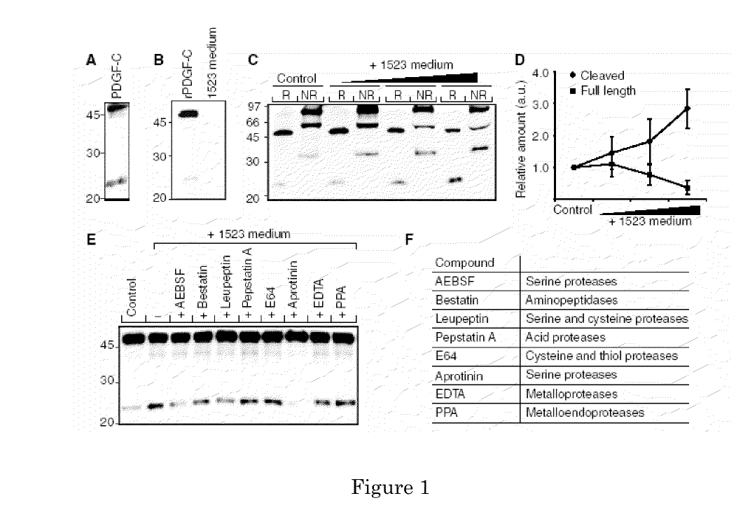

In order to identify enzymes capable of activating latent PDGF-CC, conditioned media from different in vitro-grown cell lines were screened for expression of endogenous PDGF-CC, and for the capacity to cleave and activate the secreted latent growth factor. The human fibroblastic cell line AG1523 efficiently secreted full-length PDGF-CC, and also displayed the capacity to cleave specifically full-length PDGF-C chains, thus releasing a distinct 22 kDa species under reducing conditions (FIG. 1A). This species migrated similarly to the recombinant active growth factor domain of PDGF-C expressed in insect cells (Li et al, 2000).

In an in vitro assay, the properties of the enzyme(s) involved in cleavage and activation of PDGF-CC were studied by mixing serum-free conditioned media from AG1523 cells with His6-tagged recombinant full-length PDGF-CC. Control analysis demonstrated that immunoreactivity toward the Hiss epitope was found only in recombinant PDGF-CC, and not in conditioned medium from AG1523 cells (FIG. 1B). SDS-PAGE analysis under reducing and nonreducing conditions, and immunoblotting using an anti-Hiss antibody, showed that increasing amounts of conditioned media from the AG1523 cells sequentially released the CUB domains of latent human PDGF-CC in a dose-dependent manner (FIGS. 1C and D). These data show that the enzymatic activity responsible for the cleavage of full-length PDGF-CC is derived from a secreted protease(s) present in the conditioned media from AG1523 cells.

The class of enzyme(s) responsible for cleavage and activation of latent PDGF-CC was established by generating an enzyme inhibitor profile of the enzymatic activity (FIG. 1E). Eight different protease inhibitors (see FIG. 1F) were separately preincubated with conditioned media from AG1523 cells, and then incubated with His6-tagged recombinant full-length PDGF-CC. Analysis of the incubation mixtures by SDS-PAGE and immunoblotting revealed that inhibitors of serine proteases (AEBSF, leupeptin, and aprotinin) inhibited the proteolytic cleavage of latent PDGF-CC (FIG. 1E), while inhibitors of other protease classes, including matrix metalloproteinases, failed to inhibit efficiently the processing. These results suggest that a secreted trypsin-like serine protease is responsible for the proteolytic activation of latent PDGF-CC.

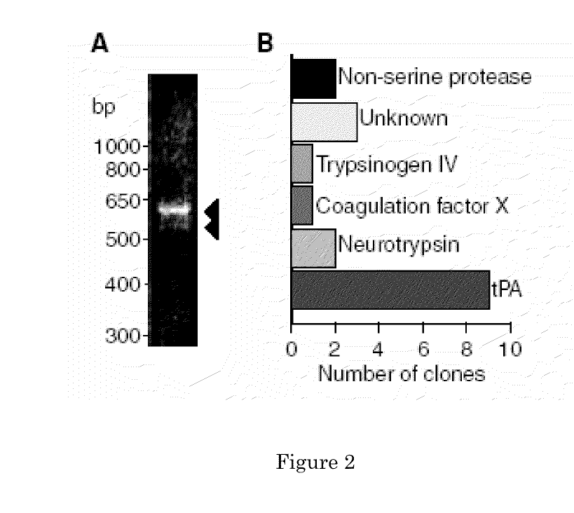

A coupled reverse transcription-polymerase chain reaction (RT-PCR) assay was employed to clone trypsin-like serine proteases expressed by AG1523 cells. Based on conserved amino-acid sequences around the catalytic triad in the serine protease domain, degenerate oligonucleotide mixtures were included in the RT-PCR reactions using single-stranded cDNA from the AG1523 cells as the template. Amplified products ranging from 500 to 650 by were visualized by agarose gel electrophoresis (FIG. 2A), subcloned, and inserts with the expected size range of approximately 550-600 by were sequenced. The results revealed that the most abundant amplified cDNA was derived from tPA, while neurotrypsin (NT), coagulation factor X, and trypsinogen IV were other known serine proteases expressed by the AG1523 cells (FIG. 2B).

### Example 2

### tPA is a Specific Activator of Latent PDGF-CC

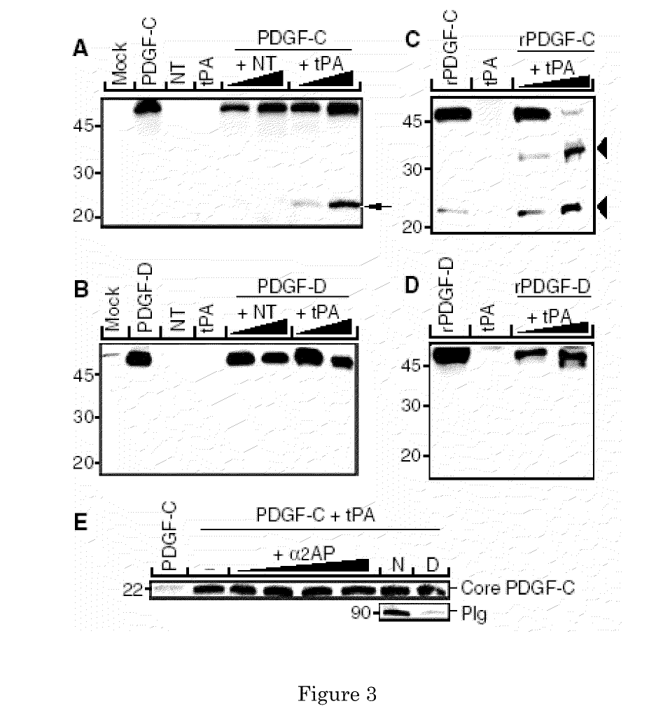

A cotransfection assay was established to identify serine proteases able to cleave and activate latent PDGF-CC. Expression plasmids encoding the relevant enzymes and full-length PDGF-C were cotransfected into COS-1 cells, and aliquots of the conditioned media from the transfectants were subjected to SDS-PAGE and immunoblotting using antibodies to the growth factor domain of PDGF-C. The results showed that tPA released the growth factor domain of latent PDGFCC, and the fragment migrated as a 22 kDa species under reducing conditions (FIG. 3A). In contrast, neurotrypsin (NT) lacked proteolytic activity toward latent PDGF-CC. As a specificity control, the ability of tPA and NT to use full-length PDGF-DD as the substrate in the cotransfection assay was analysed. The results revealed that neither of the two enzymes was able to cleave and activate latent PDGF-DD (FIG. 3B). Using purified tPA and recombinant latent PDGF-CC, or recombinant latent PDGF-DD, in an in vitro assay, these observations were confirmed showing that PDGF-CC, but not PDGF-DD, is a substrate for tPA (FIGS. 3C and D). One difference in the latter results, as compared with the results from the cotransfection assay, was that purified tPA generated a second intermediate species of 32 kDa using latent PDGF-CC as the substrate. It is possible that this intermediate is the result of digestion by plasmin contamination in the tPA preparation, since the size of the fragment is similar to that of plasmincleaved PDGF-CC previously reported (Li et al, 2000).

To ensure that the cleavage of PDGF-C observed in the cotransfection assay was a direct effect of tPA, and not an indirect effect due to cleavage by remnants of plasmin, the COS-1 cells were cultured in the absence or presence of the specific plasmin inhibitor α2-anti-plasmin or in Plg-depleted medium prior to transfection (FIG. 3E). Neither α2-antiplasmin treatment nor culturing in Plg-depleted medium had any effect on the processing of PDGF-C, showing that the cleavage of PDGF-C is performed by tPA directly.

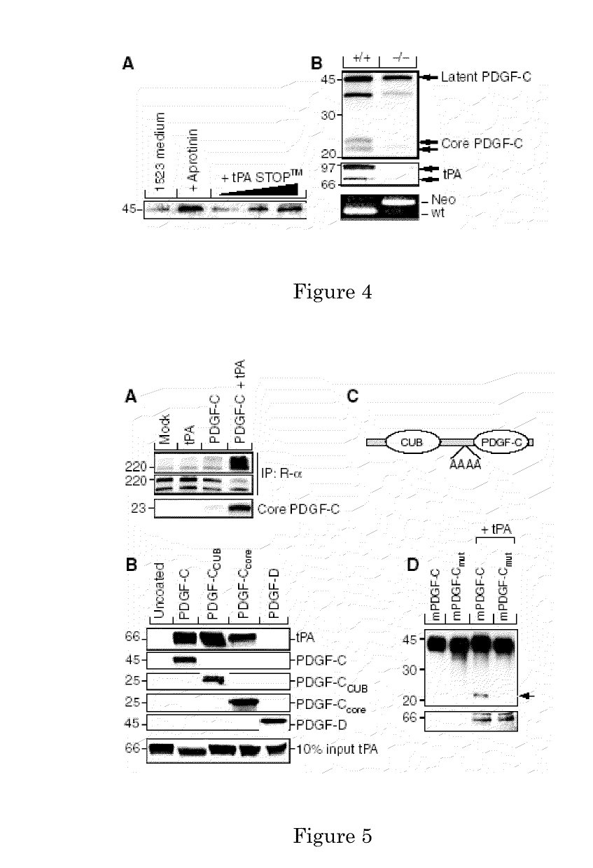

To demonstrate that the proteolytic activity of tPA accounted for the major PDGF-CC processing activity produced by AG1523 cells, a well-characterized inhibitor of tPA, tPA-STOP™ (Sturzebecher et al, 1997), and the serine protease inhibitor aprotinin (see above) were added to the serum-free culture medium of growing AG1523 cells. Analysis of conditioned media showed that tPA-STOP™, in a dose-dependent way, prevented processing of full-length PDGF-CC (FIG. 4A). Similarly, aprotinin efficiently inhibited processing of latent PDGF-CC in comparison with the untreated control. These results showed that tPA accounts for a majority of the PDGF-CC processing activity in conditioned media from AG1523 cells.

The ability of primary cultures of lung and kidney fibroblasts from wild-type and tPA-deficient mice to produce and activate latent PDGF-CC was examined. SDS-PAGE and immunoblotting analyses of TCA-precipitated proteins from serum-free conditioned media showed that the primary fibroblasts secreted latent PDGF-CC migrating as a 48 kDa species in SDS-PAGE under reducing conditions (FIG. 4B). In the medium from wild-type cells, processing of latent PDGF-CC into species migrating as 35 kDa species and as double bands of 22-25 kDa was seen. In contrast, in medium from tPA-deficient cells, the generation of double species migrating as 22-25 kDa was reduced to less than 10%, and the intensity of the 35 kDa species was also significantly reduced. These data demonstrate an essential role of tPA in activation of latent PDGF-CC in vivo.

### Example 3

### tPA-Mediated Activation of PDGF-CC Generates a PDGFR-α Agonist

It was verified that the growth factor domain in PDGF-CC released by tPA-mediated proteolysis is an efficient PDGFR-α ligand. Conditioned media from transfected COS-1 cells were applied onto porcine aortic endothelial (PAE) cells with stable expression of PDGFR-α (FIG. 5A). Stimulation of the cells using conditioned medium from mock-transfected COS-1 cells, or media from transfected COS-1 cells separately expressing tPA, or latent PDGF-CC, failed to induce receptor activation measured as induction of receptor tyrosine phosphorylation. In contrast, stimulation using medium from COS-1 cells coexpressing tPA and full-length PDGF-CC induced strong PDGFR-α activation. This showed that the growth factor domain of full-length PDGF-CC released by tPA is a bona fide ligand and activator of PDGFR-α.

The possibility of a direct protein-protein interaction between tPA and latent PDGF-CC was explored by developing a pull-down assay. Ni-NTA beads were allowed to bind recombinant Hiss-tagged latent PDGF-CC or PDGF-DD, and purified tPA was added and incubated. Following extensive washings, bound proteins were subsequently eluted with an imidazole-containing buffer, and the eluates were analyzed by immunoblotting using specific antibodies. The results showed that full-length PDGF-CC-coated beads specifically bound tPA, while uncoated Ni-NTA beads or PDGF-DD-coated beads failed to do so (FIG. 5B). Similar experiments using Ni-NTA beads separately coated with recombinant ‘free’ CUB domain or recombinant core domain of PDGF-CC showed that both domains were able to interact with tPA.

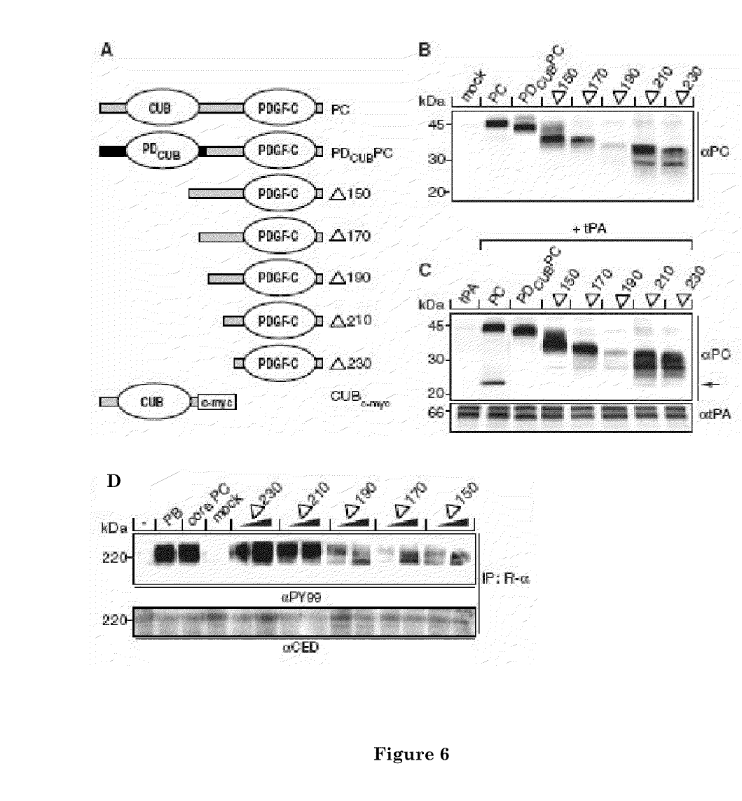

The structural requirements for recognition of full-length PDGF-CC as a substrate for tPA were mapped by analysis of several mutated forms of PDGF-CC using the co-transfection assay. The mutants of PDGF-CC included a chimeric form of PDGF-C carrying the CUB domain from PDGF-D and the hinge region and growth factor domain of PDGF-C (mutant PD_{CUB}PC), and several truncation mutants lacking the CUB domain and increasing parts of the hinge region (schematically illustratrated in FIG. 6A). All mutants were properly expressed in transfected COS-1 cells, formed disulfide-linked dimers (data not shown), and were efficiently secreted, except truncation mutant Δ190 that was expressed at a lower level in the conditioned medium (FIG. 6B). When co-transfected with tPA, neither chimeric PD_{CUB}PC, nor the truncation mutants lacking the CUB domain, were efficiently cleaved (FIG. 6C). This indicated that the CUB domain was necessary for efficient proteolytic cleavage of latent PDGF-CC by tPA.

To understand the structural requirements for receptor-binding and activation of PDGF-CC, the series of truncated mutants of PDGF-CC generated above were analysed for their ability to activate PDGFR-α in PAE cells. Conditioned media containing the truncated mutants of PDGF-CC were applied onto PAE cells, and the activation of the receptors was monitored by induction of receptor tyrosine phosphorylation (FIG. 6D). The results showed that mutants 4230 and 4210 efficiently activated PDGFR-α, while mutants with additional parts of the hinge region separating the CUB and the growth factor domains in PDGF-CC, failed to efficiently induce receptor activation. These data suggest that the cleavage site for tPA must be located within the last 40 amino acids of the hinge region upstream of the growth factor domain.

A conserved site of four amino acids containing three basic amino-acid residues (amino-acid residues -R-K-S-R-) was previously identified as a potential site for proteolytic activation of latent PDGF-CC (Li et al, 2000). It is notable that the corresponding regions in PDGF-A and PDGF-B are the cleavage sites for furine-like proteases that act in the exocytic pathway during secretion of these PDGFs (Oestman et al, 1992; Siegfried et al, 2003). To verify this, a mutant with the tribasic site replaced with alanine residues was created (schematically illustrated in FIG. 5C). Analysis using the cotransfection assay verified that the mutant was resistant to tPA-mediated cleavage, while the wild-type PDGF-CC was readily cleaved (FIG. 5D). These data suggest that tPA cleaves latent PDGF-CC in, or at least around, the conserved tribasic site.

### Example 4

### tPA-Dependent Activation of Latent PDGF-CC Drives Proliferation of Primary Fibroblasts

It was observed that primary fibroblasts derived from tPA-deficient mice grew more slowly in culture than fibroblasts derived from wild-type animals, raising the possibility that activation of latent PDGF-CC by tPA generated autocrine and paracrine growth stimulatory loops for primary fibroblasts in culture. To analyze this effect, isolated wild-type and tPA-deficient fibroblasts were serum-starved overnight, and the growth of the cells during the next 24 hours was monitored using an enzyme-based viability assay (see Example 8, Materials and Methods). The results confirmed the initial observation and showed that tPA-deficient cells displayed a reduced growth rate in serum-free medium as compared to wild-type cells (FIG. 7A). Rescue of the tPA-deficient cells by the addition of 50 ng/ml of activated PDGF-CC or recombinant tPA to the serum-free culture medium allowed the cells to grow similar to the wild-type fibroblasts.

To further demonstrate that growth of primary fibroblasts in culture was dependent on a tPA-mediated growth stimulatory loop, serum-starved fibroblast cultures were labeled with 5-bromo-2′-deoxyuridine (BrdU) for 24 hours in order to identify dividing cells. Cell nuclei were visualized with 4′,6-diamidine-2′-phenylindole dihydrochloride (DAPI), and BrdU-labeled cells were determined by immunofluorescence using antibodies to BrdU (FIG. 7B). Quantification of the results showed that the fraction of BrdU-labeled nuclei were significantly higher in wild-type fibroblasts as compared to the tPAdeficient cells (FIG. 7C). Addition of 50 ng/ml of activated PDGF-CC or recombinant tPA strongly stimulated BrdU incorporation in the tPA-deficient cells but had less effect on wild-type cells. These data suggest that autocrine and paracrine growth stimulatory loops are present in primary fibroblasts, and that these loops are generated by a tPA-mediated activation of latent PDGF-CC.

It is known that constitutive activation of PDGFRs by PDGFs leads to receptor desensitization (Heldin and Westermark, 1999), and therefore it was investigated whether the differences observed in growth between the wild-type and tPA-deficient fibroblasts upon PDGF-CC treatment were due to differential activation of PDGFR-α. Recombinant PDGF-CC protein was applied onto the primary fibroblasts and receptor activation was measured as induction of PDGFR-α tyrosine phosphorylation (FIG. 7D, upper panel). Stimulation of PDGFR-α was more pronounced in the tPA-deficient cells as compared to wild type, which might explain the efficient stimulation of proliferation seen in these cells following PDGF-CC treatment.

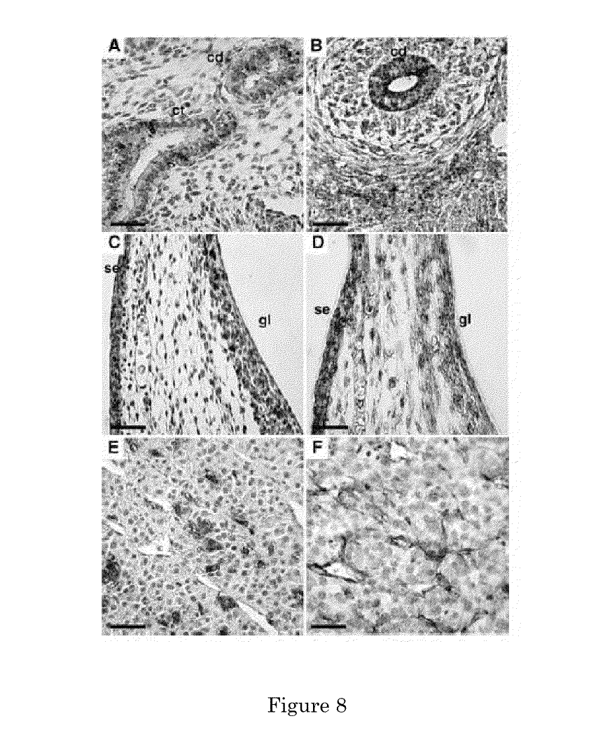

The expression patterns of PDGF-C and tPA in developing mouse embryos were compared by immunohistochemistry to examine if the two proteins were coexpressed, or expressed in adjacent cells. Expression data on tPA and PDGF-C from previously published papers also was compiled. The results of these comparisons are summarized in Table 1. Some of the results using tissue sections from E14.5 mouse embryos and T241 tumor xenografts (FIG. 8). Furthermore, the expressions of PDGF-C and tPA reported in previous publications were compiled and compared (Carroll et al, 1994; Ding et al, 2000; Aase et al, 2002). The results from these analyses suggest that PDGF-C and tPA are coexpressed in several locations in the developing embryo such as the kidney and the surface ectoderm of the skin (FIG. 8A-D). In tumor tissue sections, PDGF-C expression was observed mostly in tumor cells located at the center of the tumor in close apposition to larger blood vessels, while tPA was mainly expressed in the endothelium of the tumor blood vessels (FIGS. 8E and F). Scattered PDGF-C-positive tumor cells were also seen at the edge of the tumor. These observations support the inventors' findings and suggest that PDGF-CC can be activated by tPA in vivo.

TABLE 1
Comparison of the expression patterns of PDGF-C and tPA during embryonic development.

| | PDGF-C^{a} | tPA^{b} |
|---|---|---|
| Axial structures | | |
| Somites | Myotome | Myotome |
| Neural tube and | Notochord, sclerotome, mesenchyme surrounding | Ventral wall/floorplate of the spinal cord, |
| developing brain | the cavaties, ventral horn of the spinal cord, | dermonyotome and selected sclerotome, postmitotic |
| | floorplate | neurons of the midbrain |
| Limb bud | Surface ectoderm, interdigital mesenchyme | Surface ectoderm, core interdigital mesenchyme |
| Skeleton muscle | Developing muscle, perichondral mesenchyme, | Developing muscle |
| | hypertrophic chondrocytes | |
| Skin and derivatives | | |
| Embryonic integument | Epidermis | Epidermis |
| Hair follicle | Root sheath | Sensory hair follicle |
| Sense organs | | |
| Oral cavity | Epithelium | ND |
| Otic vesicle | Inner ear epithelium | Mesenchyme around the otic vesicle (pinna of the |
| | | ear) |
| Nasal/vomeronasal | Olfactory epithelium lining the nasal cavity | ND |
| Eye | Corneal epithelium, boundary of the eyelid | Inner layer of the optic cup |
| Whiskers | Outer layer of sheath cells | Primordium of the vibrrissae (sensory whiskers) |
| Others | | |
| Salivary gland | Epithelium and surrounding mesenchyme | ND |
| Trachea | Epithelium and surrounding mesenchyme | Epithelium and surrounding mesenchyme |
| Esophogus | Epithelium and surrounding mesenchyme | ND |
| Lung | Lung epithelium and mesenchyme | ND |
| Heart | Cardiomyocyte | Cardiac valve anlage |
| Kidney | Mesonephric ducts and tubules, metanephric | Metanephric mesenchyme, epithelium of the |
| | mesenchyme, epithelium of the collecting ducts | collecting ducts |
| Gastro-intestinal tract | Mucosal epithelium, surrounding mesenchyme, | Gut endoderm and associated mesoderm |
| | ciucular and longitudinal smooth muscle layer | |

^{a}Aase K, Mechanisms of Development 110 (2002)187-191
^{b}Carroll P M, Development 120, 3173-3183 (1994)
ND = not determined

### Example 5

### Inhibition of PDGF-CC Processing by tPa Using Antibodies Directed Against the Processing Site in PDGF-CC

This example provides a method for inhibiting proteolytic processing of PDGF-CC by tPA using antibodies directed against the -R²³¹-K²³²-S²³³-R²³⁴- cleavage site in human PDGF-C.

Sub-confluent COS-1 cells are co-transfected with expression constructs encoding tPA (pSG5-tPA, Fredriksson et al., 2004) and latent PDGF-C (pSG5-PDGF-C, Li et al., 2000) using LipofectaminePlus (LifeTechnology). 48 hrs post-transfection, the transfection medium is replaced by DMEM supplemented with polyclonal rabbit Igs (10-100 μg/ml) directed against a synthetic peptide derived from the PDGF-C sequence, extending over the cleavage site of PDGF-C (amino acids 230-250, sequence CGRSKRVVDLNLLTEEVRLYSC (SEQ ID NO: 1), the cleavage site is in bold). As a control, DMEM supplemented with an equal concentration of preimmune polyclonal rabbit Ig, is used. The conditioned serum-free medium is collected after an additional 24 hrs, and proteins are TCA precipitated as previously described (Li et al., 2000). The precipitates are subjected to SDS-PAGE under reducing conditions, immunoblotted and visualized by chemiluminiscence. PDGF-C is detected using affinity-purified polyclonal rabbit antibodies against full-length PDGF-C (Li et al., 2000) and tPA using sheep polyclonal antibodies against human tPA (ab9030, Abcam). Inhibition of PDGF-C processing and activation is monitored as diminished formation of the active 22 kDa species (Fredriksson et al. 2004).

### Example 6

### Treating Diabetic Ulcers with tPA Using a Mouse Model

An impaired wound healing model, essentially as described by Sprugel et al. ((1991) in Clinical and Experimental Approaches to Dermal and Epidermal Repair: Normal and Chronic Wounds (Barbul, A. , et al., eds) , pp. 327-340, Wiley-Liss, Inc., New York) is used. Briefly, a 1-cm-square full-thickness wound is made by excising the skin and panniculus carnosus over the paravertebral area at mid-dorsum of 15-week-old female C57BLKS/J/M++LepRdb mice (The Jackson Laboratories, Bar Harbor, ME) with glycosuria. The wound and surrounding skin is immediately covered with a self-adhesive semi-occlusive wound dressing, Bioclusive (Johnson & Johnson, Arlington, Tex.). A suitable amount of tPA, PDGF-CC, or sterile PBS vehicle, is applied to the wounds once daily for 8 days. The cut edge of each wound is traced onto a transparency sheet for planimetric analysis of wound closure on days 0 and 8. Wound areas are determined planimetrically using OPTIMAS image analysis software (Bioscan, Edmonds, Wash.). Wound closure is calculated from the wound areas by the method of Greenhalgh et al. (Greenhalgh, D. G., Sprugel, K. H., Murray, M. J., and Ross, R. (1990) Am. J. Pathol. 136, 1235-1246). The wound tissues are harvested and then embedded in paraffin for processing, and 5-μm sections are taken through the center of each wound. The sections are stained with hematoxylin and eosin for analysis. The histologic scoring system outlined by Greenhalgh et al. is followed. Minimal evidence of healing in the wound bed receives a score of 1 and a completely healed wound receives a score of 4.

This model demonstrates the novel utility of tPA or PDGF-CC in the treatment of wounds such as those arising in patients with diabetes.

### Example 7

### Free CUB Domains Act as Competitive Inhibitor in tPA-Mediated Proteolytic Activation of PDGF-CC

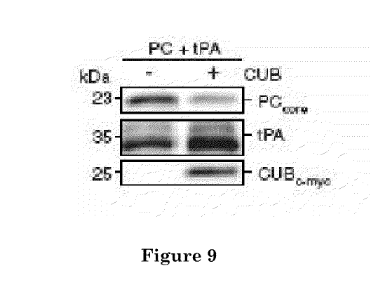

By over-expression of the “free” CUB domain of PDGF-C in the co-transfection assay, the present inventors demonstrated that the CUB domain efficiently competed for the interaction and processing of latent PDGF-CC by tPA (FIG. 9). These data suggest that both the CUB and core domains of PDGF-C directly interact with tPA, and also that the free CUB domain may act as a competitive inhibitor in tPAmediated proteolytic activation of PDGF-CC.

### Example 8

### Materials and Methods

1. Cell Culture

All cells used were maintained in Dulbecco's modified Eagle's medium (DMEM) supplemented with 10% fetal calf serum (FCS), 2mM glutamine, 100U/ml penicillin, and 100 μg/ml streptomycin, except PAE cells that were kept in supplemented F12 medium. The cells were cultured at 37° C. in a humidified 5% CO₂ atmosphere. Kidney and lung primary fibroblast cultures were prepared essentially as described (Eghbali et al, 1991) from 5-week-old wild-type (+/+) and tPA-deficient (−/−) mice (Carmeliet et al, 1994) (kindly provided by Prof. P Carmeliet, Leuven). In short, kidneys and lungs were dissected, washed in ice-cold PBS, cut into smaller pieces, and incubated with trypsin/collagenase in PBS for 20 min at 37° C. Dissociated cells were pelleted and plated. Experiments were performed on cells at passages 4-7.

2. Protein Expression and Immunoblotting

To test the endogenous expression of PDGF-CC, subconfluent AG1523 cells and primary fibroblast cultures were cultured in serum-free DMEM overnight. Recombinant Hiss-tagged human PDGF-CC species and full-length PDGF-DD were expressed in serum-free medium from Sf9 insect cells using the baculovirus expression system as described previously (Li et al, 2000; Bergsten et al, 2001). To explore the extracellular proteolytic activities in conditioned serum-free AG1523 medium, the medium was coincubated with recombinant latent PDGF-CC-containing medium (ratios 1:2, 3:2, and 10:2) at 37° C. overnight. To identify PDGF-CC activating serine proteases, the protease expression constructs were cotransfected with full-length PDGF-C (Li et al, 2000), full-length PDGF-D (Bergsten et al, 2001), or PDGF-C cleavage site mutant constructs into subconfluent COS-1 cells using LipofectaminePlus in serum-free DMEM (Life Technology). In other experiments, COS-1 cells were maintained and cultured as the AG1523 cells described above. The protease expression constructs were co-transfected with full-length PDGF-C (Li et al., 2000), with or without CUBc-myc, full-length PDGF-D (Bergsten et al., 2001), PDGF-C deletion mutants, chimeric PD_{CUB}PC or PDGF-C clevage site mutant constructs into sub-confluent COS-1 cells using Lipofectamine plus reagent according to the manufacturer's protocol (Life technology, 2 μg DNA per well in 6-well plates). Mock transfection with empty vectors served as negative control. After 24 hours the transfection medium was replaced by DMEM only. Transfection with empty vectors served as negative control. After 24 hours, the transfection medium was replaced by DMEM only, with or without the addition of α2-anti-plasmin (10 ng-1 μg, #4030, American Diagnostica Inc.), for an extra 24 hours. In addition, the COS-1 cells were grown in DMEM supplemented with 10% Plg-depleted FCS prior to transfection. Plg was removed from the FCS by affinity chromatography on lysine-Sepharose (Deutsch and Mertz, 1970) and the Plg-depleted FCS was tested by immunoblotting with rabbit anti-human Plg (A0081, DAKO). The conditioned serum-free medium was collected, and proteins were TCA precipitated as described previously (Li et al, 2000). In the case of the primary cultures, total protein concentration was measured and normalized (Bradford, 1976). All precipitates were subjected to SDS-PAGE under reducing conditions if not stated otherwise, immunoblotting, and visualization by chemiluminescence. PDGF-C and PDGF-D were detected by immunoblotting using affinity-purified polyclonal rabbit antibodies against PDGF-C (Li et al, 2000) and PDGF-D (Bergsten et al, 2001), respectively. The Hiss-tagged proteins were detected using an anti-His monoclonal antibody (C-terminal, Invitrogen). tPA was detected using sheep polyclonal antibodies against human tPA (ab9030, Abcam).

CUB_{c-myc} was detected using a rabbit affinity-purified polyclonal antibody against a human c-Myc (A-14) peptide (sc-789, Santa Cruz). Bound antibodies were visualized as above.

3. Reverse Biochemistry

All protease inhibitors were purchased from Sigma and the concentrations used were as follows: AEBSF 1 mM, bestatin 100 μM, leupeptin 100 μM, pepstatin A 10 μM, E64 100 μM, aprotinin 100 μM (-3 TIU), EDTA 50 mM, and phosphoramidon 100 μM. The protease inhibitors were preincubated with conditioned AG1523 medium at room temperature for 30 min, and then incubated with recombinant PDGF-CC (ratio 10:2) at 37° C. overnight. Recombinant PDGF-CC species were analyzed by immunoblotting as above. To determine whether tPA is the major proteolytic enzyme responsible for the PDGF-CC processing in AG1523 conditioned medium, AG1523 cells were cultured in serum-free medium, with or without the addition of a synthetic tPA inhibitor tPA-STOP™ (3.5-35 μM, #544, American Diagnostica Inc.) or 100 μM aprotinin as a positive control. The conditioned serum-free medium was collected, and proteins were precipitated before SDS-PAGE and immunoblotting using antibodies against PDGF-C (see above).

4. Cloning of Serine Proteases and Plasmid Construction

To clone trypsin-like serine proteases in AG1523 fibroblastic cells, total cellular RNAwas prepared using the guanidinium thiocyanate/acid phenol method (Chomczynski and Sacchi, 1987). Singlestranded cDNA was synthesized using AMV Reverse Transcriptase (Amersham) and oligo-dT to prime the reaction. Degenerate oligonucleotide primers flanking the conserved histidine and serine residues in the catalytic triad were designed as follows: 5′-CAR TGG GTN YTN WCN GCN GCN CAY TG (SEQ ID NO: 2) (corresponding to the amino acid sequence Q W V L/F S/T A A H C, forward) and 5′-NCC NCC NGA RTC NCC YTG RCA NGC RTC (SEQ ID NO: 3) (corresponding to the amino-acid sequence D A C Q G D S G G (SEQ ID NO: 4), reverse). The oligonucleotides were used to prime PCRs utilizing cDNA from the AG1523 cells as template. The PCR products were cloned into the pCR2.1-TOPO vector (TOPO TA Cloning kit, Invitrogen) and clones of the expected size of 500-600 by were sequenced.

Full-length human tPA was amplified by PCR using cDNA from the AG1523 cells as template and the 1750-bp product was subcloned into the pCR2.1-TOPO vector. The primers used, including a BamH1 site (underlined), were as follows: 5′-CGGG ATCCGCCGTGAATTTAAGGGAC (SEQ ID NO: 5) (forward) and 5′-CGGGATCCTTG CTTTTGAGGAGTCGG (SEQ ID NO: 6) (reverse). The BamH1 fragment was excised and cloned into the eukaryotic expression vector pSG5.

The nucleotide sequences encoding the various PDGF-CC deletion mutants, the CUB chimeric construct (PDCUBPC), the CUB domain of PDGF-C (CUBc-myc) and the cleavage site mutant were amplified by PCR using gene specific primers (shown in Table 2). All constructs were verified by sequencing. The PCR fragments of the PDGF-CC deletion mutants were excised with HindIII-EcoRI and cloned in-frame with the signal sequence of the eukaryotic expression vector pSeqTag2B (Invitrogen). The amplified PDCUBPC fragments of the CUB region (residues 1 to 172) of PDGF-D and the hinge/core region of PDGF-C (residues 166 to 345) were excised with EcoR1 and ligated. The ligation was used as template to amplify the full chimeric construct (1125 bp) (using the forward CUB and the reverse hinge/core primers). The full-length PCR product was subcloned into the pCR2.1-TOPO vector, excised with BamH1 and cloned into the eukaryotic expression vector pSG5. The CUBc-myc PCR product (residues 1 to 165) was directionally cloned into the EcoRI-BamHI sites of pSG5. To generate the clevage site mutant, mouse PDGF-C cDNA was used as template.

TABLE 2

Mutant nomenclature and description of gene specific primers used.

| Mutant | | |
|---|---|---|
| name | Description | Oligonucleotides |
| ΔN230 | PDGF-CC deletion mutant | Sense: 5′-CCCAAGCTTAGAAAATCCAGAGTG-3′ |
| | | (SEQ ID NO: 15) |
| | | Antisense: 5′-GGAATTCCTCCTGTGCTCCCTCTG-3′ |
| | | (SEQ ID NO: 16) |
| ΔN210 | PDGF-CC deletion mutant Sense: | 5′-CCCAAGCTTGACTTAGAAGATC-3′ |
| | | (SEQ ID NO: 17) |
| | | Antisense: 5′-GGAATTCCTCCTGTGCTCCCTCTG-3′ |
| | | (SEQ ID NO: 18) |
| ΔN190 | PDGF-CC deletion mutant Sense: | 5′-CCCAAGCTTACTGCCTTTAGTACC-3′ |
| | | (SEQ ID NO: 19) |
| | | Antisense: 5′-GGAATTCCTCCTGTGCTCCCTCTG-3′ |
| | | (SEQ ID NO: 20) |
| ΔN170 | PDGF-CC deletion mutant Sense: | 5′-CCCAAGCTTGTGAGTCCTTCAGTG-3′ |
| | | (SEQ ID NO: 21) |
| | | Antisense: 5′-GGAATTCCTCCTGTGCTCCCTCTG-3′ |
| | | (SEQ ID NO: 22) |
| ΔN150 | PDGF-CC deletion mutant Sense: | 5′-CCCAAGCTTCCTTCTGAACCAGGG-3′ |
| | | (SEQ ID NO: 23) |
| | | Antisense: 5′-GGAATTCCTCCTGTGCTCCCTCTG-3′ |
| | | (SEQ ID NO: 24) |
| PDCUBPC | CUB region of PDGF-DD Sense: | 5′-GCGGATCCTCCCAAATGCACCGGCTC-3′ |
| | | (SEQ ID NO: 25) |
| | | Antisense: 5′-GCGAATTCATCTTCCAGCAAAGAATA-3′ |
| | | (SEQ ID NO: 26) |
| | Hinge/core region of PDGF-CC | Sense: 5′-GCGAATTCACAGAAGCTGTGA-3′ |
| | | (SEQ ID NO: 27) |
| | | Antisense: 5′-GCGGATCCAGAATCAGCCACTGCACT-3′ |
| | | (SEQ ID NO: 28) |
| CUBc-myc | CUB domain of PDGF-CC | Sense: 5′-GCGAATTCTGAGCTCTCACCCCAGTC-3′ |
| | (including a human c-myc | (SEQ ID NO: 29) |
| | encoding sequence) | Antisense: 5′- |
| | | GCGGATCCTTACAAGTCTTCTTCAGAAATAAGCTTTTGTTCTGGCA |
| | | TGACAATGTT-3′ |
| | | (SEQ ID NO: 30) |
| Clevage | N-terminal fragment of PDGF-CC | Sense: 5′-GAATTCAGCCAAATGCTCCTCCTCGGCCTC-3′ |
| site | cleavage mutant (alanine | (SEQ ID NO: 31) |
| mutant | replacement in bold) | Antisense: 5′-TGCCGCGGCCGCCCCATACAGGAAAGCCTT-3′ |
| | | (SEQ ID NO: 32) |
| | C-terminal fragment of PDGF-CC | Sense: 5′-GCGGCCGCGGCAGTGGTGAATCTGAATCTCCTC-3′ |
| | cleavage mutant (alanine | (SEQ ID NO: 33) |
| | replacement in bold) | Antisense: 5′-GCTCTAGACTGCAGTTACCCTCCTGCGTT-3′ |
| | | (SEQ ID NO: 34) |

The fully sequenced MGC clone containing the 5′ part of human NT in the pOTB7 vector was purchased from Research Genetics whereas the 3′ part was amplified by PCR using AG1523 cDNAs as template. The primers used were as follows: 5′-GAGCTGAATACA TACGTG (SEQ ID NO: 7) (forward) and 5′-GCAGATCTGCTGCTTTGAAGTTTCCA (SEQ ID NO: 8) (reverse, including a BglII site, underlined). The resulting 1400-bp 3′ fragment was subcloned into the pCR2.1-TOPO vector and then excised with Ndel-BglII. A full-length cDNA for hNT was constructed by fusing the excised 3′ fragment with NdeI-BglIIdigested 5′-hNT/pOTB7. The full-length cDNA for hNT was excised and directionally cloned into the EcoRI-BglII sites of the eukaryotic expression vector pSG5.

To generate the cleavage site mutant, mouse PDGF-C cDNA was used as template. The predicted processing site in murine PDGF-C, amino-acid residues -K-K-S-K-, was replaced by four alanines. The N-terminal fragment of PDGF-C, containing an EcoRI and a NotI site (underlined), and the C-terminal fragment, containing a NotI and an XbaI site (underlined), were amplified using the following primers: 5′-GGAATTCAGCCAAATGCTCCTCCTCGGCCTC (SEQ ID NO: 9) (forward, N-terminal) and 5′-TGCCGCGGCCGCCCCATACAGGAAAGCCTT (SEQ ID NO: 10) (reverse, N-terminal, alanine replacement in bold), 5′-GCGGCCGC GGCAGTGGTGAATCTGAATCTCCTC (SEQ ID NO: 11) (forward, C-terminal, alanine replacement in bold), and 5′-GCTCTAGACTGCAGTTACCCTC CTGCGTT (SEQ ID NO: 12) (reverse, C-terminal). The amplified fragments were ligated and cloned in-frame into pcDNA3.1 (+) expression vector.

To produce recombinant CUB domain of human PDGF-C using the baculovirus system, the sequence encoding amino-acid residues 23-163 of PDGF-C was amplified by PCR. Primers used were as follows:

(SEQ ID NO: 13)

5′-CGGGATCCCGAATCCAACCTGAGTAG
(forward, including a BamHI site for in-frame
cloning)
and

(SEQ ID NO: 14)

5′-CCGGAATTCCTAATGGTGATGGTGATGATGTTTGTCATCGTCGTC
GACAATGTTGTAGTG
(reverse, including an EcoRI site and sequences
encoding a C-terminal His₆ tag).

The amplified product was cloned into the baculovirus expression vector pAcGP67A.

All primers used were purchased from Invitrogen and all the constructs were verified by nucleotide sequencing. The nucleotide and amino-acid sequences of human tPA can be found in the GenBank under accession number NM_{—}000930 and of hNT under accession number NM_{—}003619. The MGC clone containing the 5′ part of hNT has GenBank accession ID BC007761.

5. In vitro Cleavage and Protein-Protein Interaction Studies

Recombinant latent PDGF-CC and PDGF-DD were digested with human tPA in 100mM Tris-HCl pH 7.5, 0.1% Tween 20, and 0.1 mg/ml CNBr activated fibrinogen (Sigma) for 4 hours at 37° C. using 0.2-20 μg/ml tPA purified from human melanoma cells (T7776, Sigma). The digestions were analyzed by SDS-PAGE under reducing conditions and immunoblotted using affinity-purified antibodies against PDGF-C and PDGF-D, respectively (see above).

To determine a direct protein-protein interaction between tPA and PDGF-CC, His6-tagged recombinant protein species were bound to Ni-NTA-agarose (Qiagen) and then incubated with 1 μg of purified tPA for 2 hours at room temperature. Uncoated and PDGF-DD coated Ni-NTA beads were used as controls. The beads were washed thoroughly, and His6-tagged proteins were specifically eluted with 400 mM imidazole. Eluted proteins were analyzed by SDS-PAGE under reducing conditions and immunoblotted with antibodies against human tPA (see above). The membranes were subsequently stripped and reprobed with specific antibodies.

6. Receptor Activation and Proliferation Analysis

To monitor growth factor-induced tyrosine phosphorylation of PDGFR-α, serum-starved PAE cells stably expressing human PDGFR-α were incubated for 120 min on ice with conditioned medium from COS-1 cells transfected with full-length PDGF-C in the absence or presence of tPA. Alternatively, primary wild-type and tPA-deficient fibroblasts were stimulated with 100 ng/ml activated PDGF-CC protein. The cells were lysed as described previously (Li et al, 2000) and PDGFR-α was immunoprecipitated using a specific antiserum (Eriksson et al, 1992). Precipitated proteins were separated by SDS-PAGE under reducing conditions. Tyrosinephosphorylated receptors were detected by immunoblotting using an antiphosphotyrosine antibody (PY99, Santa Cruz). The membranes were stripped and reprobed using a polyclonal antibody against the C-terminal of the PDGFRs (CED) to detect receptor expression levels.

To monitor cell growth, both the cell proliferation reagent WST-1 (Roche) and BrdU (Sigma) were used. A total of 0.4×10⁴ (WST-1) or 1×10⁴ (BrdU) wild-type and tPA-deficient fibroblasts were seeded in triplicate-hexaplicate, and after attachment they were serum-starved overnight. Serum-starved cells were counted (WST-1 seeding control) and alternatively incubated for 24 hours in serum-free medium supplemented with 1 mg/ml BSA, and 50 μM BrdU in the BrdU experiment, in the absence or presence of 50 ng/ml activated PDGF-CC or tPA protein (#116, American Diagnostica Inc.). Upon counting, WST-1 reagent was added and measured according to the manufacturer's protocol using an ELISA reader. In the BrdU experiment, the cells were fixed in 4% paraformaldehyde in PBS for 30 min at room temperature and the DNA was denatured in 2M HCl for 20 min at room temperature and then blocked in 0.5% BSA, 0.5% Tween, and 10% goat serum in PBS. BrdU was localized by a monoclonal anti-BrdU antibody (DAKO), and proliferating cells were visualized by an Alexa594-conjugated mouse secondary antibody (Molecular Probe). To visualize all nuclei, DAPI (1 μg/ml, Roche) was included in the secondary antibody solution. Quantification of the BrdU-positive cells was performed by counting all cells along the vertical and horizontal diameters of all wells.

7. Immunohistochemical Analysis of PDGF-C and tPA Expression

Expression analysis of PDGF-C and tPA was performed by immunohistochemistry using tissue sections from E14.5 mouse embryos and T241 tumor xenografts generated from syngenic mice essentially as described previously (Aase et al, 2002). The primary antibodies used were affinity-purified rabbit antibodies directed against human PDGF-C and rabbit anti-mouse tPA IgG (#387, American Diagnostica Inc.). As negative controls, the sections were incubated only with secondary Ig or preimmune rabbit IgG, and in all cases only background staining was observed.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

### REFERENCES

Aase K, Abramsson A, Karlsson L, Betsholtz C, Eriksson U (2002) Expression analysis of PDGF-C in adult and developing mouse tissues. Mech Dev 110: 187-191.
Andrae J, Molander C, Smits A, Funa K, Nister M (2002) Plateletderived growth factor-B and -C and active a-receptors in medulloblastoma cells. Biochem Biophys Res Commun 296: 604-611 Bergsten E, Uutela M, Li X, Pietras K, Oestman A, Heldin C H, Alitalo K, Eriksson U (2001) PDGF-D is a specific, protease-activated ligand for the PDGF b-receptor. Nat Cell Biol 3: 512-516.
Boucher P, Gotthardt M, Li W P, Anderson R G, Herz J (2003) LRP: role in vascular wall integrity and protection from atherosclerosis. Science 300: 329-332.
Bradford M (1976) A rapid and sensitive method for the quantification of microgram quantities of protein utilizing the principle of protein-dye binding. Anal Biochem 72: 248-254.
Cao R, Bra kenhielm E, Li X, Pietras K, Widenfalk J, Oestman A, Eriksson U, Cao Y (2002) Angiogenesis stimulated by PDGF-CC, a novel member in the PDGF family, involves activation of PDGFR-α and -αβ receptors. FASEB J 16: 1575-1583.
Carmeliet P, Schoonjans L, Kieckens L, Ream B, Degen J, Bronson R, De Vos R, van den Oord J J, Collen D, Mulligan R C (1994) Physiological consequences of loss of plasminogen activator gene function in mice. Nature 368: 419-424.
Carroll P M, Tsirka S E, Richards W G, Frohman M A, Strickland S (1994) The mouse tissue plasminogen activator gene 5′ flanking region directs appropriate expression in development and a seizure-enhanced response in the CNS. Development 120: 3173-3183.
Chomczynski P, Sacchi N (1987) Single-step method of RNA isolation by acid guanidinium thiocyanate-phenol-chloroform extraction. Anal Biochem 162: 156-159.
Collen D (2001) Ham-Wasserman lecture: role of the plasminogen system in fibrin-homeostasis and tissue remodeling. Hematology (Am Soc Hematol Educ Program) 1-9.
Deutsch D G, Mertz E T (1970) Plasminogen: purification from human plasma by affinity chromatography. Science 170: 1095-1096.
Dijkmans J, Xu J, Masure S, Dhanaraj S, Gosiewska A, Geesin J, Sprengel J, Harris S, Verhasselt P, Gordon R, Yon J (2002) Characterization of platelet-derived growth factor-C (PDGF-C): expression in normal and tumor cells, biological activity and chromosomal localization. Int J Biochem Cell Biol 34: 414-426.
Ding H, Wu X, Kim I, Tam P P, Koh G Y, Nagy A (2000) The mouse Pdgfc gene: dynamic expression in embryonic tissues during organogenesis. Mech Dev 96: 209-213.
Eghbali M, Tomek R, Woods C, Bhambi B (1991) Cardiac fibroblasts are predisposed to convert into myocyte phenotype: specific effect of transforming growth factor beta. Proc Natl Acad Sci USA 88: 795-799.
Eriksson A, Siegbahn A, Westermark B, Heldin C-H, Claesson-Welsh L (1992) PDGF a- and b-receptors activate unique and common signal transduction pathways. EMBO J 11: 543-550.
Fredriksson, L., Li, H., Fieber, C., Li, X. and Eriksson, U. (2004) Tissue plasminogen activator is a potent activator of PDGF-CC. EMBO J, 23,3793-3802.
Gilbertson D G, Duff M E, West J W, Kelly J D, Sheppard P O, Hofstrand P D, Gao Z, Shoemaker K, Bukowski T R, Moore M, Feldhaus A L, Humes J M, Palmer T E, Hart C E (2001) Platelet-derived growth factor C (PDGF-C) a novel growth factor that binds to PDGF α and β receptor. J Biol Chem 276: 27406-27414.
Greenhalgh, D. G., Sprugel, K. H., Murray, M. J., and Ross, R. (1990) Am. J. Pathol. 136,1235-1246
Heldin C-H, Westermark B (1999) Mechanism of action and in vivo role of platelet-derived growth factor. Physiol Rev 79: 1283-1316 Hoylaerts M, Rijken D C, Lijnen H R, Collen D (1982) Kinetics of the activation of plasminogen by human tissue plasminogen activator-role of fibrin. J Biol Chem 257: 2912-2919.
Kwaan H C (1992) The plasminogen-plasmin system in malignancy. Cancer Metast Rev 11: 291-311.
LaRochelle W J, Jeffers M, McDonald W F, Chillakuru R A, Giese N A, Lokker N A, Sullivan C, Boldog F L, Yang M, Vernet C, Burgess C E, Fernandes E, Deegler L L, Rittman B, Shimkets J, Shimkets R A, Rothberg J M, Lichenstein H S (2001) PDGF-D, a new protease-activated growth factor. Nat Cell Biol 3: 517-521.
Li H, Fredriksson L, Li X, Eriksson U (2003) PDGF-D is a potent transforming and angiogenic growth factor. Oncogene 22: 1501-1510.
Li X, Eriksson U (2003) Novel PDGF family members: PDGF-C and PDGF-D. Cytokine Growth Factor Rev 14: 91-98.
Li X, Ponte'n A, Aase K, Karlsson L, Abramsson A, Uutela M, Baekstroem G, Hellstro m M, Bostro m H, Li H, Soriano P, Betsholtz C, Heldin C-H, Alitalo K, stman A, Eriksson U (2000) PDGF-C is a new protease-activated ligand for the PDGFa receptor. Nat Cell Biol 2: 302-309.
Lokker N A, Sullivan C M, Hollenbach S J, Israel M A, Giese N A (2002) Platelet-derived growth factor (PDGF) autocrine signaling regulates survival and mitogenic pathways in glioblastoma cells: evidence that the novel PDGF-C and PDGF-D ligands may play a role in the development of brain tumors. Cancer Res 62: 3729-3735.
Lyon et al., 2003, Effect of plasminogen activator inhibitor-1 in diabetes mellitus and cardiovascular disease. Am J Med. 115 Suppl 8A:62S-68S
Nakamura F, Goshima Y (2002) Structural and functional relation of neuropilins. Adv Exp Med Biol 515: 55-69 Nicole O, Docagne F, Ali C, Margaill I, Carmeliet P, MacKenzie E T, Vivien D, Buisson A (2001) The proteolytic activity of tissueplasminogen activator enhances NMDA receptor-mediated signaling. Nat Med 7: 59-64.
Oestman A, Thyberg J, Westermark B, Heldin C-H (1992) PDGF-AA and PDGF-BB biosynthesis: proprotein processing in the Golgi complex and lysosomal degradation of PDGF-BB retained intracellularly. J Cell Biol 118: 509-519.
Ranby M (1982) Studies on the kinetics of plasminogen activation by tissue plasminogen activator. Biochim Biophys Acta 704: 461-469.
Rickles R J, Strickland S (1988) Tissue plasminogen activator mRNA in murine tissues. FEBS Lett 229: 100-106.
Siegfried G, Khatib A M, Benjannet S, Chretien M, Seidah N G (2003) The proteolytic processing of pro-platelet-derived growth factor-A at RRKR(86) by members of the proprotein convertase family is functionally correlated to platelet-derived growth factor-A-induced functions and tumorigenicity. Cancer Res 63: 1458-1463.
Sprugel, K. H., Greenhalgh, D. G., Murray, M. J., and Ross, R (1991) in Clinical and Experimental Approaches to Dermal and Epidermal Repair: Normal and Chronic Wounds (Barbul, A., et al., eds), pp. 327-340, Wiley-Liss, Inc., New York
Strickland S (2001) Tissue plasminogen activator in nervous system function and dysfunction. Thromb Haemost 86: 138-143.
Sturzebecher J, Prasa D, Hauptmann J, Vieweg H, Wikstrom P (1997) Synthesis and structure-activity relationships of potent thrombin inhibitors: piperazides of 3-amidinophenylalanine. J Med Chem 40: 3091-3099.
Takeuchi T, Shuman M A, Craik C S (1999) Reverse biochemistry: use of macromolecular protease inhibitors to dissect complex biological processes and identify a membrane-type serine protease in epithelial cancer and normal tissue. Proc Natl Acad Sci USA 96: 11054-11061.
The National Institute of Neurological Disorders and Stroke rtPA Stroke Study Group (1995) Tissue plasminogen activator for acute ischemic stroke. N Engl J Med 333: 1581-1587.
Thielens N M, Bersch B, Hernandez J F, Arlaud G J (1999) Structure and functions of the interaction domains of Clr and Cls: keystones of the architecture of the C1 complex. Immunopharmacology 42: 3-13.
Tsirka S E (2002) Tissue plasminogen activator as a modulator of neuronal survival and function. Biochem Soc Trans 30: 222-225.
Tsirka, S. E., Gualandris, A., Amaral, D. G. and Strickland, S. (1995) Excitotoxininduced neuronal degeneration and seizure are mediated by tissue plasminogen activator. Nature, 377, 340-344.
Uutela M, Lauren J, Bergsten E, Li X, Horelli-Kuitunen N, Eriksson U, Alitalo K (2001) Chromosomal location, exon structure, and vascular expression patterns of the human PDGFC and PDGFD genes. Circulation 103: 2242-2247.
van Hinsbergh, V. W., Kooistra, T., Emeis, J. J. and Koolwijk, P. (1991) Regulation of plasminogen activator production by endothelial cells: role in fibrinolysis andlocal proteolysis. Int J Radiat Biol, 60, 261-272.
van Zonneveld, A. J., Chang, G. T., van den Berg, J., Kooistra, T., Verheijen, J. H., Pannekoek, H. and Kluft, C. (1986a) Quantification of tissue-type plasminogen activator (t-PA) mRNA in human endothelial-cell cultures by hybridization with a t-PA cDNA probe. Biochem J, 235, 385-390.
van Zonneveld, A. J., Veerman, H. and Pannekoek, H. (1986b) Autonomous functions of structural domains on human tissue-type plasminogen activator. Proc Natl Acad Sci USA, 83, 4670-4674.
Vassalli J D, Sappino A P, Belin D (1991) The plasminogen activator/plasmin system. J Clin Invest 88: 1067-1072.
Wang, Y. F., Tsirka, S. E., Strickland, S., Stieg, P. E., Soriano, S. G. and Lipton, S. A. (1998) Tissue plasminogen activator (tPA) increases neuronal damage after focal cerebral ischemia in wild-type and tPA-deficient mice. Nat Med, 4, 228-231.
Wu Y P, Siao C J, Lu W, Sung T C, Frohman M A, Miley P, Bugge T H, Degen J L, Levine J M, Margolis R U, Tsirka S E (2000) The tissue plasminogen activator (tPA)/plasmin extracellular proteolytic system regulates seizure-induced hippocampal mossy fiber outgrowth through a proteoglycan substrate. J Cell Biol 148: 1295-1304.
Yepes M, Sandkvist M, Coleman T A, Moore E, Wu J Y, Mitola D, Bugge T H, Lawrence D A (2002) Regulation of seizure spreading by neuroserpin and tissue-type plasminogen activator is plasminogen-independent. J Clin Invest 109: 1571-1578.
Yepes M, Sandkvist M, Moore E G, Bugge T H, Strickland D K, Lawrence D A (2003) Tissue-type plasminogen activator induces opening of the blood-brain barrier via the LDL receptor-related protein. J Clin Invest 112: 1533-1540.
Zwerner J P, May W A (2001) PDGF-C is an EWS/FLI induced transforming growth factor in ewing family tumors. Oncogene 20: 626-633.

1-32. (canceled)

33. A pharmaceutical composition for inhibiting proteolytic processing of PDGF-C or PDGF-CC in a mammal in need thereof, comprising an effective amount of a substance which an antibody that binds specifically to the proteolytic cleavage site of PDGF-C and inhibits proteolytic processing of PDGF-C, and a pharmaceutically suitable excipient.

34-37. (canceled)

38. The composition according to claim 33, wherein the antibody is raised using a polypeptide having a sequence of CGRSKRVVDLNLLTEEVRLYSC.

39-65. (canceled)

66. An antibody against the tPA processing site (RSKR) on PDGF-C or PDGF-CC, which antibody inhibits activation of PDGF-C or PDGF-CC by tPA.

67. The antibody according to claim 66, wherein the antibody is raised using a polypeptide having a sequence of CGRSKRVVDLNLLTEEVRLYSC.

68. The antibody according to claim 66, wherein the antibody is a monoclonal antibody, a polyclonal antibody, a humanized, chimerized or full human antibody.

69. A fragment of the antibody according to claim 66, wherein said fragment inhibits activation of PDGF-C or PDGF-CC by tPA, and is a Fab, Fab₂, F(ab′)₂, Fv, Fc, Fd, or scFvs fragment.


SEQUENCE LISTING (34 sequences)

SEQ ID NO: 1 - protein, 22 aa, Artificial
  Partial sequence of PDGF-C
CGRSKRVVDLNLLTEEVRLYSC

SEQ ID NO: 2 - DNA, 26 bp, Artificial
  degenerate oligonucleotide primer
cartgggtnytnwcngcngcncaytg

SEQ ID NO: 3 - DNA, 27 bp, artificial
  degenerate oligonucleotide primer
nccnccngartcnccytgrcangcrtc

SEQ ID NO: 4 - protein, 9 aa, artificial
  partial sequence of PDGF-C
DACQGDSGG

SEQ ID NO: 5 - DNA, 26 bp, artificial
  oligonucleotide primer
cgggatccgccgtgaatttaagggac

SEQ ID NO: 6 - DNA, 26 bp, artificial
  oligonucleotide primer
cgggatccttgcttttgaggagtcgg

SEQ ID NO: 7 - DNA, 18 bp, artificial
  oligonucleotide primer
gagctgaatacatacgtg

SEQ ID NO: 8 - DNA, 26 bp, artificial
  oligonucleotide primer
gcagatctgctgctttgaagtttcca

SEQ ID NO: 9 - DNA, 31 bp, artificial
  oligonucleotide primer
ggaattcagccaaatgctcctcctcggcctc

SEQ ID NO: 10 - DNA, 30 bp, artificial
  oligonucleotide primer
tgccgcggccgccccatacaggaaagcctt

SEQ ID NO: 11 - DNA, 33 bp, artificial
  oligonucleotide primer
gcggccgcggcagtggtgaatctgaatctcctc

SEQ ID NO: 12 - DNA, 29 bp, artificial
  oligonucleotide primer
gctctagactgcagttaccctcctgcgtt

SEQ ID NO: 13 - DNA, 26 bp, artificial
  oligonucleotide primer
cgggatcccgaatccaacctgagtag

SEQ ID NO: 14 - DNA, 60 bp, artificial
  oligonucleotide primer
ccggaattcctaatggtgatggtgatgatgtttgtcatcgtcgtcgacaatgttgtagtg

SEQ ID NO: 15 - DNA, 24 bp, artificial
  oligonucleotide primer
cccaagcttagaaaatccagagtg

SEQ ID NO: 16 - DNA, 24 bp, artificial
  oligonucleotide primer
ggaattcctcctgtgctccctctg

SEQ ID NO: 17 - DNA, 22 bp, artificial
  oligonucleotide primer
cccaagcttgacttagaagatc

SEQ ID NO: 18 - DNA, 24 bp, artificial
  oligonucleotide primer
ggaattcctcctgtgctccctctg

SEQ ID NO: 19 - DNA, 24 bp, artificial
  oligonucleotide primer
cccaagcttactgcctttagtacc

SEQ ID NO: 20 - DNA, 24 bp, artificial
  oligonucleotide primer
ggaattcctcctgtgctccctctg

SEQ ID NO: 21 - DNA, 24 bp, artificial
  oligonucleotide primer
cccaagcttgtgagtccttcagtg

SEQ ID NO: 22 - DNA, 24 bp, artificial
  oligonucleotide primer
ggaattcctcctgtgctccctctg

SEQ ID NO: 23 - DNA, 24 bp, artificial
  oligonucleotide primer
cccaagcttccttctgaaccaggg

SEQ ID NO: 24 - DNA, 24 bp, artificial
  oligonucleotide primer
ggaattcctcctgtgctccctctg

SEQ ID NO: 25 - DNA, 26 bp, artificial
  oligonucleotide primer
gcggatcctcccaaatgcaccggctc

SEQ ID NO: 26 - DNA, 26 bp, artificial
  oligonucleotide primer
gcgaattcatcttccagcaaagaata

SEQ ID NO: 27 - DNA, 21 bp, artificial
  oligonucleotide primer
gcgaattcacagaagctgtga

SEQ ID NO: 28 - DNA, 26 bp, artificial
  oligonucleotide primer
gcggatccagaatcagccactgcact

SEQ ID NO: 29 - DNA, 26 bp, artificial
  oligonucleotide primer
gcgaattctgagctctcaccccagtc

SEQ ID NO: 30 - DNA, 56 bp, artificial
  oligonucleotide primer
gcggatccttacaagtcttcttcagaaataagcttttgttctggcatgacaatgtt

SEQ ID NO: 31 - DNA, 30 bp, artificial
  oligonucleotide primer
gaattcagccaaatgctcctcctcggcctc

SEQ ID NO: 32 - DNA, 30 bp, artificial; identical to SEQ ID NO: 10
  oligonucleotide primer

SEQ ID NO: 33 - DNA, 33 bp, artificial; identical to SEQ ID NO: 11
  oligonucleotide primer

SEQ ID NO: 34 - DNA, 29 bp, artificial
  oligonucleotide primer
gctctagactgcagttaccctcctgcgtt